SEQUENCE LISTING (29 sequences)

SEQ ID NO: 1 - DNA, 1156 bp, Bacillus lentus
cggtttcttggcctccatatactaatacgactcactatagggagaccacaagaaggagatatacatatggctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcggtaccatggggaattagccgtgtgcaagccccagctgcccataaccgtggattgacaggttctggtgtaaaagttgctgtcctcgatacagggatatccactcatccagatctaaatattcgtggtggcgcaagctttgtaccaggggaaccgtcgactcaagatgggaatgggcatggcacgcatgtggccgggacgatcgctgctttaaacaattcgattggcgttcttggcgtagctcctagcgctgagctatacgctgttaaagtcctaggggcgagcggttcaggttcggtcagctcgattgcccaaggattggaatgggcagggaacaatggcatgcacgttgctaatttgagtttaggaagcccttcgccaagtgccacactcgagcaagctgttaatagcgcgacttctagaggcgttcttgttgtagcggcatctgggaattcaggtgcaggctcaatcagctatccggcgcgctatgcgaacgcaatggcagtcggagctactgatcaaaacaacaaccgcgctagcttttcacagtatggcgcaggccttgacattgtcgcacccggggtaaacgtgcagagcacatacccaggttcaacatatgccagcttaaacggtacatcgatggctactcctcatgttgcaggtgcggccgcccttgttaaacaaaagaacccatcttggtctaatgtacaaattcgaaatcatctaaagaatacggcaactagtttaggaagcacgaacttgtatggaagcggacttgttaacgcagaagcggcaacgcgttaataactataagcgcacacgtactgac

SEQ ID NO: 2 - DNA, 1065 bp, Bacillus lentus
atggctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcggtaccatggggaattagccgtgtgcaagccccagctgcccataaccgtggattgacaggttctggtgtaaaagttgctgtcctcgatacagggatatccactcatccagatctaaatattcgtggtggcgcaagctttgtaccaggggaaccgtcgactcaagatgggaatgggcatggcacgcatgtggccgggacgatcgctgctttaaacaattcgattggcgttcttggcgtagctcctagcgctgagctatacgctgttaaagtcctaggggcgagcggttcaggttcggtcagctcgattgcccaaggattggaatgggcagggaacaatggcatgcacgttgctaatttgagtttaggaagcccttcgccaagtgccacactcgagcaagctgttaatagcgcgacttctagaggcgttcttgttgtagcggcatctgggaattcaggtgcaggctcaatcagctatccggcgcgctatgcgaacgcaatggcagtcggagctactgatcaaaacaacaaccgcgctagcttttcacagtatggcgcaggccttgacattgtcgcacccggggtaaacgtgcagagcacatacccaggttcaacatatgccagcttaaacggtacatcgatggctactcctcatgttgcaggtgcggccgcccttgttaaacaaaagaacccatcttggtctaatgtacaaattcgaaatcatctaaagaatacggcaactagtttaggaagcacgaacttgtatggaagcggacttgttaacgcagaagcggcaacgcgttaa

SEQ ID NO: 3 - protein, 354 aa, Bacillus lentus
MAEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 4 - DNA, 1156 bp, Bacillus lentus
cggtttcttggcctccatatactaatacgactcactatagggagaccacaagaaggagatatacatatggctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcggtaccatggggaattagccgtgtgcaagccccagctgcccataaccgtggattgacaggttctggtgtaaaagttgctgtcctcgatacagggatatccactcatccagatctaaatattcgtggtggcgcaagctttgtaccaggggaaccgtcgactcaagatgggaatgggcatggcacgcatgtggccgggacgatcgctgctttaaacaattcgattggcgttcttggcgtagctcctagcgctgagctatacgctgttaaagtcctaggggcgagcggttcaggttcggtcagctcgattgcccaaggattggaatgggcagggaacaatggcatgcacgttgctaatttgagtttaggaagcccttcgccaagtgccacactcgagcaagctgttaatagcgcgacttctagaggcgttcttgttgtagcggcatctgggaattcaggtgcaggctcaatcagctatccggcgcgctatgcgaacgcaatggcagtcggagctactgatcaaaacaacaaccgcgctagcttttcacagtatggcgcaggccttgacattgtcgcacccggggtaaacgtgcagagcacatacccaggttcaacatatgccagcttaaacggtacagcaatggctactcctcatgttgcaggtgcggccgcccttgttaaacaaaagaacccatcttggtctaatgtacaaattcgaaatcatctaaagaatacggcaactagtttaggaagcacgaacttgtatggaagcggacttgttaacgcagaagcggcaacgcgttaataactataagcgcacacgtactgac

SEQ ID NO: 5 - DNA, 941 bp, Bacillus lentus
atggctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcggtaccatggggaattagccgtgtgcaagccccagctgcccataaccgtggattgacaggttctggtgtaaaagttgctgtcctcgatacagggatatccactcatccagatctaaatattcgtggtggcgcaagctttgtaccaggggaaccgtcgactcaagatgggaatgggcatggcacgcatgtggccgggacgatcgctgctttaaacaattcgattggcgttcttggcgtagctcctagcgctgagctatacgctgttaaagtcctaggggcgagcggttcaggttcggtcagctcgattgcccaaggattggaatgggcagggaacaatggcatgcacgttgctaatttgagtttaggaagcccttcgccaagtgccacactcgagcaagctgttaatagcgcgacttctagaggcgttcttgttgtagcggcatctgggaattcaggtgcaggctcaatcagctatccggcgcgctatgcgaacgcaatggcagtcggagctactgatcaaaacaacaaccgcgctagcttttcacagtatggcgcaggccttgacattgtcgcacccggggtaaacgtgcagagcacatacccaggttcaacatatgccagcttaaacggtacagcaatggctactcctcatgttgcaggtgcggccgcccttgttaa

SEQ ID NO: 6 - protein, 312 aa, Bacillus lentus
MAEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTHGYSSCCRCGRPC

SEQ ID NO: 7 - DNA, 22 bp, Artificial Sequence
  Artificial DNA Primer
cggtttcttggcctccatatac

SEQ ID NO: 8 - DNA, 24 bp, Artificial Sequence
  Artificial DNA Primer
aagtcagtacgtgtgcgcttatag

SEQ ID NO: 9 - DNA, 22 bp, Artificial Sequence
  Artificial DNA Primer
gtcagtacgtgtgcgcttatag

SEQ ID NO: 10 - DNA, 90 bp, Artificial Sequence
  Artificial DNA Primer
cagaacgtcactctctcttcactaatacgactcactatagggagaccacaagaaggagatatacatatggctgaagaagcaaaagaaaaa

SEQ ID NO: 11 - DNA, 87 bp, Artificial Sequence
  Artificial DNA Primer
cagaacgtcactctctcttcactaatacgactcactatagggagaccacaagaaggagatatacatatggcgcaatcggtaccatgg

SEQ ID NO: 12 - DNA, 46 bp, Artificial Sequence
  Artificial DNA Primer
gctcacctgggctatgtattagttattaacgcgttgccgcttctgc

SEQ ID NO: 13 - DNA, 22 bp, Artificial Sequence
  Artificial DNA Primer
cagaacgtcactctctcttcac

SEQ ID NO: 14 - DNA, 24 bp, Artificial Sequence
  Artificial DNA Primer
aaaaaacggagcgaaccacttatc

SEQ ID NO: 15 - DNA, 20 bp, Artificial Sequence
  Artificial DNA Primer
ggcatgcacgttgctaattt

SEQ ID NO: 16 - DNA, 21 bp, Artificial Sequence
  Artificial DNA Primer
gctacaacaagaacgcctcta

SEQ ID NO: 17 - protein, 275 aa, Bacillus amyloliquefaciens [sequence omitted]

SEQ ID NO: 18 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 19 - protein, 274 aa, Bacillus subtilis
AQTVPYGIPLIKADKVQAQGYKGANVKVGIIDTGIAASHTDLKVVGGASFVSGESYNTDGNGHGTHVAGTVAALDNTTGVLGVAPNVSLYAIKVLNSSGSGTYSAIVSGIEWATQNGLDVINMSLGGPSGSTALKQAVDKAYASGIVVVAAAGNSGSSGSQNTIGYPAKYDSVIAVGAVDSNKNRASFSSVGAELEVMAPGVSVYSTYPSNTYTSLNGTSMASPHVAGAAALILSKYPTLSASQVRNRLSSTATNLGDSFYYGKGLINVEAAAQ

SEQ ID NO: 20 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGTYSGIVSGIEWATTNGMDVINMSLGGPSGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 21 - protein, 268 aa, Bacillus lentus
QTVPWGISFINTQQAHNRGIFGNGARVAVLDTGIASHPDLRIAGGASFISSEPSYHDNNGHGTHVAGTIAALNNSIGVLGVRPSADLYALKVLDRNGSGSLASVAQGIEWAINNNMHIINMSLGSTSGSSTLELAVNRANNAGILLVGAAGNTGRQGVNYPARYSGVMAVAAVDQNGQRASFSTYGPEIEISAPGVNVNSTYTGNRYVSLSGTSMATPHVAGVAALVKSRYPSYTNNQIRQRINQTATYLGSPSLYGNGLVHAGRATQ

SEQ ID NO: 22 - protein, 269 aa, Bacillus alcalophilus
AQSVPWGISRVQAPAAHNRGLTGSGVKVAVLDTGISTHPDLNIRGGASFVPGEPSTQDGNGHGTHVAGTIAALNNSIGVLGVAPNAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 23 - protein, 268 aa, Bacillus YaB
QTVPWGINRVQAPIAQSRGFTGTGVRVAVLDTGISNHADLRIRGGASFVPGEPNISDGNGHGTQVAGTIAALNNSIGVLGVAPNVDLYGVKVLGASGSGSISGIAQGLQWAANNGMHIANMSLGSSAGSATMEQAVNQATASGVLVVAASGNSGAGNVGFPARYANAMAVGATDQNNNRATFSQYGAGLDIVAPGVGVQSTVPGNGYASFNGTSMATPHVAGVAALVKQKNPSWSNVQIRNHLKNTATNLGNTTQFGSGLVNAEAATR

SEQ ID NO: 24 - protein, 272 aa, Bacillus sp. NKS-21
QTVPWGIPYIYSDVVHRQGYFGNGVKVAVLDTGVAPHPDLHIRGGVSFISTENTYVDYNGHGTHVAGTVAALNNSYGVLGVAPGAELYAVKVLDRNGSGSHASIAQGIEWAMNNGMDIANMSLGSPSGSTTLQLAADRARNAGVLLIGAAGNSGQQGGSNNMGYPARYASVMAVGAVDQNGNRANFSSYGSELEIMAPGVNINSTYLNNGYRSLNGTSMASPHVAGVAALVKQKHPHLTAAQIRNRMNQTAIPLGNSTYYGNGLVDAEYAAQ

SEQ ID NO: 25 - protein, 269 aa, Bacillus sp. G-825-6
NQVTPWGITRVQAPTAWTRGYTGTGVRVAVLDTGISTHPDLNIRGGVSFVPGEPSYQDGNGHGTHVAGTIAALNNSIGVVGVAPNAELYAVKVLGANGSGSVSSIAQGLQWTAQNNIHVANLSLGSPVGSQTLELAVNQATNAGVLVVAATGNNGSGTVSYPARYANALAVGATDQNNNRASFSQYGTGLNIVAPGVGIQSTYPGNRYASLSGTSMATPHVAGVAALVKQKNPSWSNTQIRQHLTSTATSLGNSNQFGSGLVNAEAATR

SEQ ID NO: 26 - protein, 279 aa, Thermoactinomyces vulgaris [sequence omitted]

SEQ ID NO: 27 - DNA, 19 bp, Artificial Sequence
  Artificial DNA Primer
gttcaacatatgccagctt

SEQ ID NO: 28 - DNA, 16 bp, Artificial Sequence
  Artificial DNA Primer
cgcacctgcaacatga

SEQ ID NO: 29 - DNA, 16 bp, Artificial Sequence
  Artificial DNA Primer
acggtacatcgatggc

(19) United States
(12) Patent Application Publication
(10) Pub. No.: US 2019/0161786 A1
(43) Pub. Date: May 30, 2019

(54) METHODS FOR SELECTING ENZYMES HAVING PROTEASE ACTIVITY

(71) Applicant: NOVOZYMES A/S, Bagsvaerd (DK)

(72) Inventors: Robert Blazej, San Francisco, CA (US); Nicholas Toriello, San Carlos, CA (US); Charles Emrich, San Francisco, CA (US)

(73) Assignee: NOVOZYMES A/S, Bagsvaerd (DK)

(21) Appl. No.: 16/091,765

(22) PCT Filed: Apr. 7, 2017
(86) PCT No.: PCT/US2017/026626

§ 371 Date: Oct. 5, 2018

Related U.S. Application Data
(60) Provisional application No. 62/319,747, filed on Apr. 7, 2016.

(51) Int. Cl. C12Q 1/37; C12N 15/10; C40B 50/06; C40B 20/02; C40B 40/08

(52) U.S. Cl. CPC C12Q 1/37; C12N 15/1075; C12N 15/1058; C40B 50/08; C40B 20/02; C40B 40/08; C40B 50/06

(57) ABSTRACT

Provided herein are systems and components thereof for improving protease activity. The systems make use of an emulsion for in vitro compartmentalization of a library of synthetic compounds, each compound having a gene linked to a protease substrate and selectable marker. Expressed enzymes with greater protease activity will preferentially hydrolyze the protease substrate, whereas enzymes with less protease activity will leave the substrate intact. Removal of the non-hydrolyzed compounds provides an enriched gene library encoding for more active protease variants. Also described are synthetic compounds and emulsions which can be used in the methods.

### REFERENCE TO A SEQUENCE LISTING

This application contains a Sequence Listing in computer readable form, which is incorporated herein by reference.

### FIELD OF THE INVENTION

The present invention is in the technical field of protein engineering design and selection. More particularly, the present invention relates to enzyme enhancement by means of directed evolution.

### BACKGROUND

Proteases are used for a variety of industrial applications, including, inter alia, household care in detergent compositions for improved stain removal. While proteases (e.g., subtilases) have been successfully modified to improve desired properties such as stability and effectiveness at lower wash temperatures (See, e.g., PCT/EP2015/078586, WO 2016/001449, and US 2015/0125925) development of protease variants typically includes protein engineering techniques, such as rational design and/or directed evolution, followed by laborious enzymatic assays to test for improved function. Thus, there is a strong need for methods of rapid and efficient identification of those synthetic genes that encode polypeptides having improved protease activity.

### SUMMARY

Described herein are systems and components thereof for improving protease activity. Accordingly, in one aspect is a method of selecting for a polypeptide having protease activity, the method comprising:

(i) suspending a plurality of synthetic compounds in an aqueous phase, wherein the synthetic compounds individually comprise:
(a) a polynucleotide encoding for a polypeptide;
(b) a protease substrate linked to said polynucleotide; and
(c) a selectable marker linked to said polynucleotide;

wherein the aqueous phase comprises components for expression of the polypeptide;

(ii) forming a water-in-oil emulsion with the aqueous phase, wherein the synthetic compounds are compartmentalized in aqueous droplets of the emulsion;

(iii) expressing the polypeptides within the aqueous droplets of the emulsion, wherein a polypeptide with protease activity in an aqueous droplet hydrolyzes the protease substrate in that droplet; and

(iv) separating the synthetic compounds to recover synthetic compounds comprising the protease substrate and/or synthetic compounds wherein the protease substrate has been hydrolyzed.

In one embodiment, the polypeptide comprises a propeptide. In another embodiment, the selectable marker is linked to the polynucleotide in a distal position relative to the protease substrate.

In another aspect is a synthetic compound, comprising: (a) a polynucleotide encoding for a polypeptide; (b) a protease substrate linked to said polynucleotide; and (c) a selectable marker linked to said polynucleotide. In one embodiment, the polypeptide comprises a propeptide. In another embodiment, the selectable marker is linked to the polynucleotide in a distal position relative to the protease substrate.

In another aspect is a method of making the synthetic compound, comprising: (i) linking a protease substrate to a polynucleotide encoding for a polypeptide; (ii) linking a selectable marker to the polynucleotide encoding for a polypeptide; and (ii) recovering the synthetic compound.

In another aspect is a polynucleotide library, comprising a plurality of the synthetic compounds.

In another aspect is a water-in-oil emulsion, comprising the polynucleotide library, wherein the synthetic compounds of the library are compartmentalized in aqueous droplets of the emulsion.

In another aspect is a method of making the emulsion, comprising: (i) suspending the plurality of synthetic compounds in the aqueous phase, and (ii) mixing the suspension of (i) with an oil.

### BRIEF DESCRIPTION OF THE FIGURES

FIG. 1 shows an exemplary diagrammatic representation of the process steps involved in protease selection in accordance with one aspect of the present invention.

FIG. 2 shows a graphical representation of the self-enrichment of wild-type protease compared to catalytically inactive protease by using the systems in the present invention.

FIG. 3 shows the yield and activity of IVTT-expressed pro- and mature protease using the systems of the present invention. Results from protease containing a propeptide are shown in dark grey and those from the mature protease are show in white. Note that both yield and activity for the mature protease are so low as to be barely visible on the chart.

FIG. 4 shows the differential capture of amplicons with either a proximal or distal biotin affinity tag linked to a synthetic compound of the present invention. Amplicon with proximal biotin is shown in grey, and amplicon with distal biotin is shown in black. “Extracted” measurement was sampled from the aqueous fraction extracted after breaking the emulsions. “Captured” measurement was sampled from DNA captured on streptavidin-coated beads.

FIG. 5 shows a graphical representation of the effect of ovoinhibitor on amount of recovered DNA by using the systems in the present invention.

### DEFINITIONS

Amino acid: The terms “amino acid” or “amino acid residue,” include naturally occurring L-amino acids or residues, unless otherwise specifically indicated. The terms “amino acid” and “amino acid residue” also include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Coding sequence: The term “coding sequence” or “coding region” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Distal/proximal position: The term “distal position” means the referenced selectable marker is linked to the opposite end of the polynucleotide relative to the referenced substrate (e.g., when the substrate is linked to the 5′ end of a strand, then a distal selectable marker is linked to the 3′ end of the same strand as the substrate, or when linked to the 5′ end of the complementary strand. Likewise, when the substrate is linked to the 3′ end of a strand, then a distal selectable marker is linked to the 5′ end of the same strand as the substrate, or when linked to the 3′ end of the complementary strand). In some embodiments, the substrate is linked to the 5′ end of a strand and the marker is in a distal position on the 5′ end of the complementary strand.

The term “proximal position” means the referenced selectable marker is linked to the same end of the polynucleotide relative to the referenced substrate (e.g., when the substrate is linked to the 5′ end of a strand, then a proximal selectable marker is linked to the 5′ end of the same strand as the substrate, or linked to the 3′ end of the complementary strand. Likewise, when the substrate is linked to the 3′ end of a strand, then a proximal selectable marker is linked to the 3′ end of the same strand as the substrate, or linked to the 5′ end of the complementary strand).

Expression: The term “expression” includes the process of producing a polypeptide from a coding sequence, and may include but is not limited to, transcription, post-transcriptional modification, translation, post-translational modification, and secretion. Expression can be measured—for example, to detect increased expression—by techniques known in the art, such as measuring levels of mRNA and/or translated polypeptide. Expression, as used herein, includes in vitro transcription/translation.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, and the like with a nucleic acid construct or expression vector comprising a polynucleotide described herein (e.g., a polynucleotide encoding a protease or protease variant). The term “host cell” encompasses any progeny of a parent cell that is not identical to the parent cell due to mutations that occur during replication.

Linker: The term “linker” or “linked”, as used herein, refers to the chemical attachment of one referenced compound to another referenced compound.

Mature polypeptide: The term “mature polypeptide” is defined herein as a polypeptide having biological activity that is in its final form following translation and any post-translational modifications, such as N-terminal processing, C-terminal truncation, glycosylation, phosphorylation, etc. In some embodiments, the mature polypeptide is amino acids 86-354 of SEQ ID NO: 3.

Mutant: The term “mutant” means a polynucleotide encoding a variant.

Nucleic acid construct: The term “nucleic acid construct” means a nucleic acid molecule, either single- or double-stranded, which comprises one or more control sequences. The construct may be isolated from a naturally occurring gene, modified to contain segments of nucleic acids in a manner that would not otherwise exist in nature, or synthetic.

Operably linked: The term “operably linked” means a configuration in which a control sequence is placed at an appropriate position relative to the coding sequence of a polynucleotide such that the control sequence directs expression of the coding sequence.

Parent or parent protease: The term “parent” or “parent protease” means a protease to which an alteration is made to produce an enzyme variant. The parent may be a naturally occurring (wild-type) polypeptide or a variant or fragment thereof.

Polynucleotide: The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers to any form of DNA or RNA, including, for example, genomic DNA; complementary DNA (cDNA), which is a DNA representation of messenger RNA (mRNA), usually obtained by reverse transcription of mRNA or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands). Polynucleotides are said to be “different” if they differ in structure, e.g., nucleotide sequence.

Polypeptide: The term “polypeptide” refers to an amino acid polymer and is not meant to refer to a specific length of the encoded product and, therefore, encompasses peptides, oligopeptides, and proteins. The polypeptide may also be a naturally occurring allelic or engineered variant of a polypeptide.

Propeptide: The term “propeptide” is an amino acid sequence linked (fused) in frame to the amino terminus of a polypeptide, wherein the resultant polypeptide is known as a proenzyme or propolypeptide (or a zymogen in some cases). A propolypeptide is generally inactive or less active and can be converted to a mature active polypeptide by catalytic or autocatalytic cleavage of the propeptide from the propolypeptide resulting in an active form of the polypeptide. In some embodiments, the propeptide is amino acids 1-85 of SEQ ID NO: 3.

Prepropeptide: The term “prepropeptide” is defined herein as a signal peptide and propeptide present at the amino terminus of a polypeptide, where the propeptide is linked (or fused) in frame to the amino terminus of a polypeptide and the signal peptide region is linked in frame (or fused) to the amino terminus of the propeptide region.

Protease: The term “protease” is defined herein as an enzyme that hydrolyses peptide bonds. It includes any enzyme belonging to the EC 3.4 enzyme group (including each of the thirteen subclasses thereof). The EC number refers to Enzyme Nomenclature 1992 from NC-IUBMB, Academic Press, San Diego, Calif., including supplements 1-5 published in Eur. J. Biochem. 223: 1-5 (1994); Eur. J. Biochem. 232: 1-6 (1995); Eur. J. Biochem. 237: 1-5 (1996); Eur. J. Biochem. 250: 1-6 (1997); and Eur. J. Biochem. 264: 610-650 (1999); respectively. The term “subtilases” refer to a sub-group of serine protease according to Siezen et al., 1991, Protein Engng. 4: 719-737 and Siezen et al., 1997, Protein Science 6: 501-523. Serine proteases or serine peptidases is a subgroup of proteases characterised by having a serine in the active site, which forms a covalent adduct with the substrate. Further the subtilases (and the serine proteases) are characterised by having two active site amino acid residues apart from the serine, namely a histidine and an aspartic acid residue. The subtilases may be divided into 6 sub-divisions, i.e. the Subtilisin family, the Thermitase family, the Proteinase K family, the Lantibiotic peptidase family, the Kexin family and the Pyrolysin family. The term “protease activity” means a proteolytic activity (EC 3.4). Proteases of the invention are endopeptidases (EC 3.4.21). Protease activity may be determined using methods known in the art (e.g., US 2015/0125925) or using commercially available assay kits (e.g., Sigma-Aldrich).

Signal peptide: The term “signal peptide” is defined herein as a peptide linked (fused) in frame to the amino terminus of a polypeptide having biological activity and directs the polypeptide into the cell's secretory pathway. A propeptide may be present between the signal peptide and the amino terminus of the polypeptide (see prepropeptide definition supra).

Substrate: As used herein, the term “substrate” generally refers to a substrate for an enzyme; i.e., the material on which an enzyme acts to produce a reaction product.

Solid phase: As used herein, a “solid phase” refers to any material that is a solid when employed in the selection methods of the invention.

Synthetic compound: As used herein, the term “synthetic compound” refers to a compound that is not naturally occurring.

Variant: The term “variant” means a protease comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more amino acids adjacent to and immediately following the amino acid occupying a position.

Wild-type protease: The term “wild-type” protease means a protease expressed by a naturally occurring microorganism, such as a bacterium, yeast, or filamentous fungus found in nature.

Reference to “about” a value or parameter herein includes aspects that are directed to that value or parameter per se. For example, description referring to “about X” includes the aspect “X”. When used in combination with measured values, “about” includes a range that encompasses at least the uncertainty associated with the method of measuring the particular value, and can include a range of plus or minus two standard deviations around the stated value.

As used herein and in the appended claims, the singular forms “a,” “or,” and “the” include plural referents unless the context clearly dictates otherwise. It is understood that the aspects described herein include “consisting” and/or “consisting essentially of” aspects.

Unless defined otherwise or clearly indicated by context, all technical and scientific terms used herein have the same meaning as commonly understood by one of ordinary skill in the art.

### DETAILED DESCRIPTION

Described herein, inter alia, are methods and components used thereof for improving protease activity. The invention employs in vitro compartmentalization (IVC) for rapid and high throughput enzyme evolution. Instead of relying on a physical link between the genotype and phenotype as implemented in display technologies, IVC links genotype and phenotype by spatial confinement in a single aqueous droplet of a water-in-oil emulsion (See, e.g., Tawfik et al., 1998, Nat. Biotechnol. 16(7): 652-656; U.S. Pat. No. 6,489,103; WO 99/02671; WO 2009/124296).

However, existing IVC screening systems have several disadvantages that make them unsuitable for screening proteases, e.g., requiring a soluble gene-linked substrate that is converted into a product that remains linked to the gene (WO 99/02671), or requiring an insoluble solid-phase cellulosic substrate (WO 2009/124296). Additionally, the Applicant has found that compartmentalization of an active protease presents challenges likely related to autolysis. The Applicant further found that the presence and location of a selective marker on the gene-linked substrate has a significant effect on the capability of screening for polypeptides with protease activity.

Accordingly, described herein is a selection method for enhancing protease activity. The method makes use of IVC and a collection of synthetic bioconjugate compounds that function as both a selection substrate and a means of encoding a protease that acts on the substrate. The collection of synthetic compounds includes a collection of polynucleotides that encode for polypeptides (in particular, protease or protease derivatives) linked to a collection of protease substrates. An expressed polypeptide having protease activity can then hydrolyze the protease substrate, followed by separation of the hydrolyzed and non-hydrolyzed synthetic compounds. Based on these methods, the Applicant further discovered that use of a protease having a propeptide sequence provides significantly enhanced yield and activity. Without being bound by theory, the propeptide sequence likely minimizes autolysis thereby allowing sufficient accumulation of expressed protease with the compartmentalized gene linked substrate. The Applicant also surprisingly discovered that location of a distal affinity tag on the gene linked substrate results in significantly enhanced recovery of the released DNA compared to an affinity tag in the proximal location.

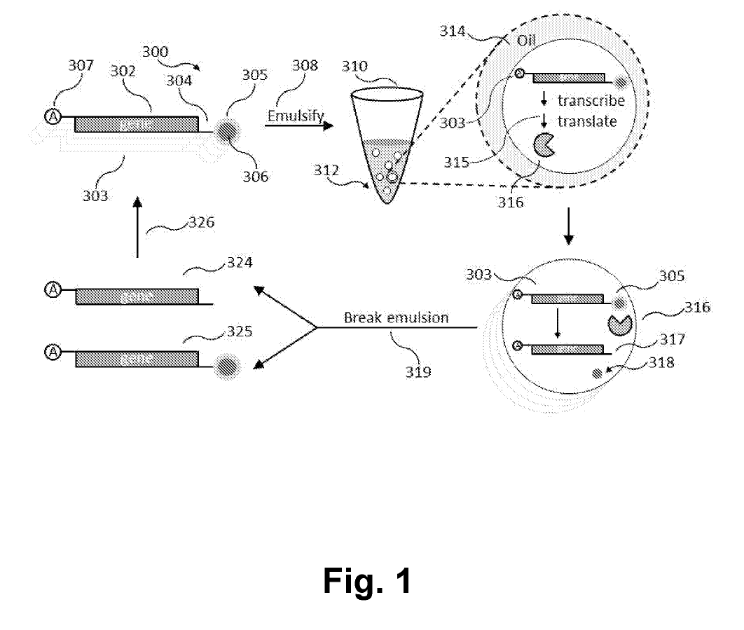

As exemplified in FIG. 1, a selection method (300) may employ a collection of polynucleotides (302) encoding for polypeptides, such as a library of synthetic compounds (303) comprising the polynucleotides. The polynucleotides of the library (302) are linked (304) to a protease substrate (305) and may be coated onto the surface of a solid phase (306) (e.g., magnetic microsphere) and typically mutants that encode variants of an enzyme having protease activity toward the substrate (305). The polynucleotide mutants (302) of the library (303) encoding for the protease variants may be created using a variety of techniques including mutagenic PCR and DNA library synthesis as set forth in more detail below. PCR amplification using a chemically-modified PCR primer provides one means of linking (304) polynucleotide mutants (302) to a protease substrate (305). The polynucleotide mutants (302) may be linked to a selectable marker (307) to provide additional means of selectively recovering released polynucleotide mutants (302) at the end of the process. The polynucleotide library (303) may be emulsified (308) using various oil-surfactants (314) with water to create an emulsion (310) containing aqueous droplets (312) (compartments), each with a compartmentalized synthetic compound. The emulsion is incubated to allow for expression (315) of the polynucleotide mutants (302) into corresponding polypeptides (316).

The expressed polypeptide variants (316) exhibiting protease activity toward the protein substrate (305) then hydrolyze the substrate (318). Protease variants with enhanced protease activity are probabilistically more likely to hydrolyze the DNA-bound protease substrate (305) than protease variants exhibiting lower activity. A variable incubation temperature and time, as well as use of inhibitors and competitive substrates, enables tuning the assay stringency. After incubation, the emulsion (310) is broken (319). The synthetic compounds which were linked to a hydrolyzed protease substrate (324) are then separated from synthetic compounds with a non-hydrolyzed protein substrate (325) using techniques described herein. Recovery of the synthetic compounds (324) may be facilitated, e.g., using affinity capture. Polynucleotide mutants that encode polypeptide variants with enhanced protease activity toward the substrate may be subjected to additional rounds (326) of selection to further enhance protease activity.

Accordingly, in one aspect is a method of selecting for a polypeptide having protease activity, the method comprising:

(i) suspending a plurality of synthetic compounds in an aqueous phase, wherein the synthetic compounds individually comprise:
(a) a polynucleotide encoding for a polypeptide;
(b) a protease substrate linked to said polynucleotide; and
(c) a selectable marker linked to said polynucleotide;

wherein the aqueous phase comprises components for expression of the polypeptide;

(ii) forming a water-in-oil emulsion with the aqueous phase, wherein the synthetic compounds are compartmentalized in aqueous droplets of the emulsion;

(iii) expressing the polypeptides within the aqueous droplets of the emulsion, wherein a polypeptide with protease activity in an aqueous droplet hydrolyzes the protease substrate in that droplet; and

(iv) separating the synthetic compounds to recover synthetic compounds comprising the protease substrate and/or synthetic compounds wherein the protease substrate has been hydrolyzed.

### Synthetic Compounds

In one aspect, the synthetic compounds used herein comprise (a) a polynucleotide encoding for a polypeptide; (b) a protease substrate linked to said polynucleotide; and (c) a selectable marker linked to said polynucleotide. In some embodiments, a synthetic compound comprises two polynucleotides (e.g., having the same or different sequence). In some embodiments, a synthetic compound comprises only one copy of one polynucleotide.

### Polynucleotides/Polypeptides

The polynucleotides may comprise a coding sequence for a polypeptide that is, or is derived from, a protease. Suitable proteases may be of fungal, bacterial, including filamentous fungi and yeast, and plant origin. Chemically modified or protein engineered mutant enzymes are included, as well as a combination of any of the sources supra or a computationally derived sequence based on evolutionary trees and/or a de novo sequence based on structure prediction.

In one embodiment, the polypeptide is (or is derived from) an acidic protease, i.e., a protease characterized by the ability to hydrolyze proteins under acidic conditions below pH 7, e.g., at a pH between 2-7. In one embodiment the acidic protease has an optimum pH in the range from 2.5 and 3.5 (determined on high nitrogen casein substrate at 0.7% w/v at 37° C.) and a temperature optimum between 5 to 50° C. at an enzyme concentration of 10 mg/mL at 30° C. for one hour in 0.1 M piperazine/acetate/glycine buffer).

In another embodiment, the polypeptide is (or is derived from) an alkaline protease, i.e., a protease characterized by the ability to hydrolyze proteins under alkaline conditions above pH 7, e.g., at a pH between 7-11. In one embodiment, the alkaline protease is derived from a strain of Bacillus, e.g., Bacillus licheniformis. In one embodiment, the alkaline protease has an optimum temperature in the range from 7 and 11 and a temperature optimum around 70° C. determined at pH 9.

In another embodiment, the polypeptide is (or is derived from) a neutral protease, i.e., a protease characterized by the ability to hydrolyze proteins under conditions between pH 5 and 8. In one embodiment, the alkaline protease is derived from a strain of Bacillus, e.g., Bacillus amyloliquefaciens. In one embodiment, the alkaline protease has an optimum pH in the range between 7 and 11 (determined at 25° C., 10 minutes reaction time with an enzyme concentration of 0.01-0.2 AU/L) and a temperature optimum between 50° C. and 70° C. (determined at pH 8.5, 10 minutes reaction time and 0.03-0.3 AU/L enzyme concentration.

In one embodiment, the polypeptide is (or is derived from) a metalloprotease. In one embodiment, the protease is derived from a strain of the genus Thermoascus, e.g., a strain of Thermoascus aurantiacus, such as Thermoaccus aurantiacus CGMCC No. 0670 having the sequence shown in the mature part of SEQ ID NO: 2 in WO 03/048353 hereby incorporated by reference. The Thermoaccus aurantiacus protease is active from 20-90° C., with an optimum temperature around 70° C. Further, the enzyme is activity between pH 5-10 with an optimum around pH 6. In some embodiments, the protease is a subtilisin from Thermoactinomyces vulgaris.

Suitable plant proteases may be derived from barley.

Suitable bacterial proteases, such as subtilases, include Bacillus proteases, derived from, e.g., Bacillus amyloliquefaciens, Bacillus lentus, Bacillus licheniformis, Bacillus subtilis, Bacillus alcalophilus. Suitable filamentous bacterial proteases may be derived from a strain of Nocardiopsis, preferably Nocardiopsis prasina NRRL 18262 protease (or Nocardiopsis sp. 10R) and Nocardiopsis dassonavilla NRRL 18133 (Nocardiopsis dassonavilla M58-1) both described in WO 1988/003947 (Novozymes).

Suitable acid fungal proteases include fungal proteases derived from Aspergillus, Mucor, Rhizomucor, Rhizopus, Candida, Corio/us, Endothia, Enthomophtra, Irpex, Penicillium, Sclerotium, Thermoaccus, and Torulopsis. Especially contemplated are proteases derived from Aspergillus niger (see, e.g., Koaze et al., 1964, Agr. Biol. Chem. Japan 28: 216), Aspergillus saitoi (see, e.g., Yoshida, 1954, J. Agr. Chem. Soc. Japan 28: 66), Aspergillus awamori (Hayashida et al., 1977, Agric. Biol. Chem. 42(5): 927-933, Aspergillus aculeatus (WO 95/02044), or Aspergillus oryzae; proteases from Mucor pusillus or Mucor miehei disclosed in U.S. Pat. No. 4,357,357 and U.S. Pat. No. 3,988,207; and Rhizomucor mehei or Rhizomucor pusillus disclosed in, e.g., WO 94/24880 (hereby incorporated by reference).

Aspartic acid proteases are described in, for example, Handbook of Proteolytic Enzymes, Edited by A. J. Barrett, N. D. Rawlings and J. F. Woessner, Academic Press, San Diego, 1998, Chapter 270). Suitable examples of aspartic acid protease include, e.g., those disclosed in Berka et al., 1990, Gene 96: 313); (Berka et al., 1993, Gene 125: 195-198); and Gomi et al., 1993, Biosci. Biotech. Biochem. 57: 1095-1100, which are hereby incorporated by reference.

The polypeptide may be a component (or derived from a component) of a commercial product, such as ALCALASE®, ESPERASE™, NEUTRASE®, RENILASE®, NOVOZYM™ FM 2.0L, and NOVOZYM™ 50006 (available from Novozymes A/S, Denmark) and GC106™ and SPEZYME™ FAN from Genencor Int., Inc., USA.

In some embodiments, the polypeptide is (or is derived from) a subtilase. In some embodiments, the polypeptide is a Bacillus lentus protease, e.g., of SEQ ID NO: 3, or derived from Bacillus lentus protease of SEQ ID NO: 3. In some embodiments, the polypeptide is a Bacillus amyloliquefaciens protease, e.g., of SEQ ID NO: 17, or derived from Bacillus amyloliquefaciens protease of SEQ ID NO: 17. In some embodiments, the polypeptide is a Bacillus subtilis 168 protease, e.g., of SEQ ID NO: 18, or derived from Bacillus subtilis 168 protease of SEQ ID NO: 18. In some embodiments, the polypeptide is a Bacillus subtilis DY protease, e.g., of SEQ ID NO: 19, or derived from Bacillus subtilis DY protease of SEQ ID NO: 19. In some embodiments, the polypeptide is a Bacillus licheniformis protease, e.g., of SEQ ID NO: 20, or derived from Bacillus licheniformis protease of SEQ ID NO: 20. In some embodiments, the polypeptide is a Bacillus lentus protease, e.g., of SEQ ID NO: 21, or derived from Bacillus lentus protease of SEQ ID NO: 21. In some embodiments, the polypeptide is a Bacillus alcalophilus PB92 protease, e.g., of SEQ ID NO: 22, or derived from Bacillus alcalophilus PB92 protease of SEQ ID NO: 22. In some embodiments, the polypeptide is a Bacillus YaB protease, e.g., of SEQ ID NO: 23, or derived from Bacillus YaB protease of SEQ ID NO: 23. In some embodiments, the polypeptide is a Bacillus sp. NKS-21 protease, e.g., of SEQ ID NO: 24, or derived from Bacillus sp. NKS-21 protease of SEQ ID NO: 24. In some embodiments, the polypeptide is a Bacillus sp. G-825-6 protease, e.g., of SEQ ID NO: 25, or derived from Bacillus sp. G-825-6 protease of SEQ ID NO: 25. In some embodiments, the polypeptide is a Thermoactinomyces vulgaris protease, e.g., of SEQ ID NO: 26, or derived from Thermoactinomyces vulgaris protease of SEQ ID NO: 26.

The polynucleotide may comprise a mutated protease coding sequence that encodes for a protease variant of a parent protease. The protease variants comprise an alteration, i.e., a substitution, insertion, and/or deletion, at one or more (e.g., several) positions. Examples of protease variants are described in WO 2016/087617, WO 2016/001449, and US 2015/0125925, the content of which is incorporated herein by reference.

The polynucleotide may comprise a sequence that encodes for a propeptide. For example, the wild-type Bacillus lentus protease of SEQ ID NO: 3 comprises the propeptide amino acid sequence 1-85 linked in frame to the amino terminus of a mature protease (amino acids 86-354). The Applicant has demonstrated in the Examples below that polynucleotides encoding a polypeptide comprising a propeptide are surprisingly well-suited for the present invention. Thus, it is contemplated by the Applicant that in any embodiment described herein, the polypeptide further comprises a propeptide sequence. The polynucleotide may further code for a signal sequence (e.g., to slow self-maturation of the pro-protein) either fused directly to the amino terminus of the mature peptide sequence, or with the a propeptide (a prepropeptide) which is fused directly to the amino terminus of the mature peptide sequence. In some embodiments, the polynucleotide does not encode a signal sequence.

The polynucleotides may comprise suitable control sequences, such as those required for efficient expression of the gene product, for example promoters, enhancers, translational initiation sequences, polyadenylation sequences, splice sites and the like, and as described in detail below.

As described supra, the methods of the present invention may comprise a plurality of synthetic compounds to create a polynucleotide library (e.g., a polynucleotide library encoding a library of protease variants). In particular embodiments, the libraries have at least about: 10²,10³,10⁴, 10⁵, 10⁶, 10⁷, 10⁸, 10⁹, 10¹⁰, 10¹¹, 10¹², or 10¹⁴ different synthetic compounds and/or polynucleotides. Generally, the size of the library will be less than about 10¹⁵.

Libraries of polynucleotides can be created in any of a variety of different ways that are well known to those of skill in the art. In particular, pools of naturally occurring polynucleotides can be cloned from genomic DNA or cDNA (Sambrook et al., 1989; Molecular cloning: a laboratory manual. Cold Spring Harbor Laboratory Press, New York); for example, phage antibody libraries, made by PCR amplification repertoires of antibody genes from immunized or unimmunized donors have proved very effective sources of functional antibody fragments (Winter et al., 1994; Annu. Rev. Immunol. 12: 433-55; and Hoogenboom, 1997, Trends Biotechnol. 15: 62-70). Libraries of genes can also be made by encoding all (see for example Smith, 1985, Science 228: 1315-1317; and Parmley and Smith, 1988, Gene 73: 305-318) or part of genes (see for example Lowman et al., 1991, Biochemistry 30: 10832-10838) or pools of genes (see for example Nissim et al., 1994, Embo J. 13: 692-698) by a randomized or doped oligonucleotide synthesis.

Libraries can also be made by introducing mutations into a polynucleotide or pool of polynucleotides randomly by a variety of techniques in vivo, including; using mutator strains, of bacteria such as E. coli mutD5 (Liao et al., 1986, Proc. Natl. Acad. Sci. USA 83: 576-580; Yamagishi et al., 1990, Protein Eng. 3: 713-719; Low et al., 1996, J. Mol. Biol., 260: 359-368); using the antibody hypermutation system of B-lymphocytes (Yelamos et al., 1995, Nature 376: 225-229). Random mutations can also be introduced both in vivo and in vitro by chemical mutagens, and ionizing or UV irradiation (see Friedberg et al., 1995, DNA repair and mutagenesis. ASM Press, Washington D.C.), or incorporation of mutagenic base analogues (Freese, 1959, J. Mol. Biol. 1: 87; Zaccolo et al., 1996, J. Mol. Biol. 255: 589-603). Random mutations can also be introduced into genes in vitro during polymerization for example by using error-prone polymerases (Leung et al., 1989, Technique 1: 11-15). Further diversification can be introduced by using homologous recombination either in vivo (see Kowalczykowski et al., 1994, Microbiol. Rev. 58: 401-65) or in vitro (Stemmer, 1994, Nature 370: 389-391; and Stemmer, 1994, Proc. Natl. Acad. Sci. USA 91: 10747-10751)). Libraries of complete or partial genes can also be chemically synthesized from sequence databases or computationally predicted sequences.

Libraries can also be made using DNA recombination like, e.g., DNA shuffling. Shuffling between two or more homologous input polynucleotides (starting-point polynucleotides) involves fragmenting the polynucleotides and recombining the fragments, to obtain output polynucleotides (i.e. polynucleotides that have been subjected to a shuffling cycle) wherein a number of nucleotide fragments are exchanged in comparison to the input polynucleotides. DNA recombination or shuffling may be a (partially) random process in which a library of chimeric genes is generated from two or more starting genes. A number of known formats can be used to carry out this shuffling or recombination process. The process may involve random fragmentation of parental DNA followed by reassembly by peR to new full-length genes, e.g. as presented in U.S. Pat. No. 5,605,793; U.S. Pat. No. 5,811,238; U.S. Pat. No. 5,830,721; U.S. Pat. No. 6,117,679. In-vitro recombination of genes may be carried out, e.g. as described in U.S. Pat. No. 6,159,687; WO 98/41623; U.S. Pat. No. 6,159,688; U.S. Pat. No. 5,965,408; U.S. Pat. No. 6,153,510. The recombination process may take place in vivo in a living cell, e.g. as described in WO 97/07205 and WO 98/28416. The parental DNA may be fragmented by DNase I treatment or by restriction endonuclease digests as described by Kikuchi et al (2000, Gene 236:159-167). Shuffling of two parents may be done by shuffling single stranded parental DNA of the two parents as described in Kikuchi et al. (2000, Gene 243:133-137). A particular method of shuffling is to follow the methods described in Crameri et al., 1998, Nature 391: 288-291 and Ness et al., Nature Biotechnology 17: 893-896. Another format would be the methods described in U.S. Pat. No. 6,159,687: Examples 1 and 2.

### Substrates and Selectable Markers

The protease substrates used with the synthetic compounds described herein may be any suitable substrate as determined by the skilled artisan based on the desired protease activity and/or other desired properties of the selection methods. For example, the substrate may be any suitable substrate for the proteases described supra, including but not limited to substrates for acidic proteases, alkaline proteases, neutral proteases, metalloproteases, subtilases, and aspartic acid proteases, such as α-casein, bovine serum albumin, hemoglobin, keratin, ovalbumin. In some embodiments, the substrate is cross-linked and/or heat-treated as known in the art.

The selectable marker may be any suitable marker which may be used in a biochemical assay to distinguish and/or recover those compounds that have been altered by an active protease using the methods of the invention described herein.

Suitable selectable markers include, but are not limited to affinity tags, wherein each affinity tag is a member of a binding pair. When used in the methods described herein, an affinity tag can further aid in separation of hydrolyzed and non-hydrolyzed substrate in step (iv), as the hydrolyzed compounds, e.g., may be released from a solid phase and selectively removed from the mixture by affinity capture following removal of the non-hydrolyzed compounds using standard techniques.

Examples of binding pairs that may be used in the present invention include an antigen and an antibody or fragment thereof capable of binding the antigen, the biotin avidin/streptavidin pair (Savage et al., 1994, Avidin-biotin chemistry: a handbook. Pierce Chemical Company, Rockford), a calcium-dependent binding polypeptide and ligand thereof (e.g., calmodulin and a calmodulin-binding peptide (Stofko et al., 1992, FEBS Lett. 302: 274-278; Montigiani et al.,1996, J. Mol. Biol. 258: 6-13)), pairs of polypeptides which assemble to form a leucine zipper (Tripet et al., 1996, Protein Engng. 9: 1029-1042), histidines (typically hexahistidine peptides) and chelated Cu²⁺, Zn²⁺ and Ni²⁺, (e.g. Ni-NTA; Hochuli et al., 1987, J. Chromatogr. 411: 177-84), RNA-binding and DNA-binding proteins (Klug, 1995, Ann. NY Acad. Sci. 758: 143-60) including those containing zinc-finger motifs (Klug and Schwabe, 1995, Faseb T. 9: 597-604) and DNA methyltransferases (Anderson, 1993, Curr. Op. Struct. Biol. 3: 24-30), and their nucleic acid binding sites. For example, suitable affinity tags include, inter alia, biotin, digoxigenin, dinitrophenyl (DNP), fluorescein, rhodamine (e.g., Texas Red®), and fucose. Biotin and fucose are capable of binding avidin and lectin, respectively, whereas digoxigenin, DNP, fluorescein, and rhodamine are capable of binding to product-specific antibodies. In one embodiment, the synthetic compound comprises a biotin selectable marker. In this embodiment, the synthetic compounds wherein the protease substrate has been hydrolyzed of step (iv) may be separated with streptavidin (e.g., streptavidin coated microspheres).

As noted supra, the Applicant surprisingly discovered that location of a distal affinity tag on the gene linked substrate results in significantly enhanced recovery of the released DNA compared to an affinity tag in the proximal location. Accordingly, in one embodiment, the selectable marker (e.g., affinity tag) is linked to the polynucleotide in a distal position relative to the protease substrate.

Conjugation of the protease substrate and selectable marker may be carried out using a variety of available conjugation techniques and preferably does not interfere with gene expression or the activity of the enzyme on the substrate. Standard synthetic techniques may be employed, such as coupling the substrate or affinity tag with the polynucleotide using a reactive handle (e.g., an activated ester, azide, maleimide, etc.). In one example, a free hydroxyl of a marker or substrate can be coupled to a maleimide-linked oligonucleotide primer. The resulting conjugate is then amplified by PCR with a template polynucleotide sequence to generate the desired synthetic compound. In another example, a 5′-thiol primer is coupled to a marker or substrate modified with a maleimide moiety, prior to PCR amplification to afford the desired synthetic compound. Similarly, an amino group on a modified marker, substrate or polynucleotide can be linked to an activated ester (e.g., an NHS-ester) to produce the desired synthetic compound. Even further still, the conjugation can employ click chemistry, for example, wherein an azide-modified marker or substrate is conjugated to an oligonucleotide primer having (i) a terminal alkyne for a copper(l) catalyzed [3+2] azide-alkyne cycloaddition (CuAAC), or (ii) a cyclooctyne derivative, such as dibenzocyclooctyl (DBCO), for a Cu-free click cycloaddition (Jewett et al., 2010, Chem. Soc. Rev. 39(4):1272). Accordingly, in some embodiments, the selectable marker and/or substrate is linked to the polynucleotide with a substituted thiol (e.g., thioether), substituted amino (e.g., amido), or triazole moiety.

### Solid Phases

The synthetic compounds described herein may further comprise a solid phase. Materials useful as solid phases can include: natural polymeric carbohydrates and their synthetically modified, crosslinked, or substituted derivatives, such as agar, agarose, cross-linked alginic acid, chitin, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins, and keratins; natural hydrocarbon polymers, such as latex and rubber; synthetic polymers, such as vinyl polymers, including polyethylene, polypropylene, polystyrene, polyvinylchloride, polyvinyl acetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

Solid phases generally have a size and shape that permits their suspension in an aqueous medium, followed by formation of a water-in-oil emulsion. Suitable solid phases include microbeads or particles (both termed “microparticles” for ease of discussion). Microparticles useful in the invention can be selected by one skilled in the art from any suitable type of particulate material and include, but are not limited, to those composed of cellulose, Sepharose, polystyrene, polymethylacrylate, polypropylene, latex, polytetrafluoroethylene, polyacrylonitrile, polycarbonate, or similar materials.

In some embodiments, the solid phase is the protease substrate (e.g., a particle that is both a solid phase and a protease substrate).

In some embodiments, the solid phase is a hydrophobic microbead (e.g., silica beads coated with C4, C8, and C18 alkyl groups, polystyrene, or PS-divinyl benzene). The use of hydrophobic solid phases may further enable separation of the synthetic compounds in step (iv), since compounds that remain attached to the solid phase will more likely be found in the oil phase whereas compounds that have been cleaved from the solid phase by hydrolysis will more likely be found in the aqueous phase.

Preferred microparticles include those averaging between about 0.01 and about 35 microns, more preferably between about 0.5 to about 20 microns in diameter, or about 0.1 to about 5 microns in diameter, haptenated microparticles, microparticles impregnated by one or preferably at least two fluorescent dyes (particularly those that can be identified after individual isolation in a flow cell and excitation by a laser), ferrofluids (i.e., magnetic particles less than about 0.1 micron in size), magnetic micro spheres (e.g., superparamagnetic particles about 3 microns in size), and other microparticles collectable or removable by sedimentation and/or filtration.

In some embodiments, the solid phase is a nanoparticle, such as a gold nanoparticle. Also contemplated are solid lipid nanoparticles, e.g., as described by Ekambaram et al., 2012, Sci. Revs. Chem. Commun. 2(1): 80-102. The nanoparticles are generally between about 1 to 400 nm in average diameter (e.g., 1 to 100 nm) and include, e.g., spherical colloidal gold, gold nanorods, and urchian shaped nanoparticles.

The solid phases are linked to the synthetic compounds by any means known to those in the art that do not interfere with expression of the linked polynucleotides. For example, an amine modified synthetic compound may be linked to tosyl or carboxylate modified microspheres. Likewise, amino modified microspheres may be coupled to a tosyl or carboxylate modified synthetic compound (or to an amino modified synthetic compound via glutaraldehyde). Hydroxyl, hydrazide or chloromethyl modified microspheres can also be employed, as known in the art. Exemplary synthetic methods for linking compounds to gold nanoparticles can be found in PCT/US2016/026441.

In some embodiments, the solid phase is linked to the protease substrate, thereby anchoring the protease substrate to the solid phase. For example, a whole protein substrate which can be conjugated to pre-activated solid phase beads (e.g., epoxide or tosyl-activated magnetic polystyrene beads, such as Dynal M270 or M280). In these embodiments, the synthetic compound may be cleaved from the solid phase in the methods of the invention by hydrolysis of an active protease.

Also contemplated are methods of making the synthetic compounds described herein, comprising: (i) linking a protease substrate to a polynucleotide encoding for a polypeptide; (ii) linking a selectable marker to the polynucleotide encoding for a polypeptide; and (ii) recovering the synthetic compound. In some embodiments wherein the synthetic compound comprises a solid phase, the method further comprises linking the protease substrate to a solid phase.

### Formation of Aqueous Phases Containing Reagents for Polypeptide Expression

Synthetic compounds are combined in an aqueous phase with components for expression of the polypeptide (e.g., in vitro transcription/translation). Such components can be selected for the requirements of a specific system from the following: a suitable buffer, an in vitro transcription/replication system and/or an in vitro translation system containing all the necessary ingredients, enzymes and cofactors, RNA polymerase, nucleotides, transfer RNAs, ribosomes and amino acids (natural or synthetic).

A suitable buffer typically allows the desired components of the biological system to be active and will therefore depend upon the requirements of each specific reaction system. Buffers suitable for biological and/or chemical reactions are known in the art and recipes provided in various laboratory texts, such as Sambrook et al., 1989 (supra).

Exemplary in vitro translation systems can include a cell extract, typically from bacteria (Zubay, 1973, Annu. Rev. Genet. 7: 267-87; Zubay, 1980, Methods Enzymol. 65: 856-877; Lesley et al., 1991, J. Biol. Chem. 266(4): 2632-2638; Lesley, 1995, Methods Mol. Biol. 37: 265-278), rabbit reticulocytes (Pelham and Jackson, 1976, Eur. J. Biochem. 67: 247-256), or wheat germ (Anderson et al., 1983, Methods Enzymol. 101: 635-44). Many suitable systems are commercially available (for example from Promega) including some which will allow coupled transcription/translation (all the bacterial systems and the reticulocyte and wheat germ TNT.™. extract systems from Promega). The mixture of amino acids used may include synthetic amino acids if desired, to increase the possible number or variety of proteins produced in the library. This can be accomplished by charging tRNAs with artificial amino acids and using these tRNAs for the in vitro translation of the proteins to be selected (Ellman et al., 1991, Methods Enzymol. 202: 301-336; Benner, 1994, Trends Biotechnol. 12: 158-63; Mendel et al., 1995, Annu. Rev. Biophys. Biomol. Struct. 24: 435-462).

As exemplified below, the aqueous phase may further comprise a protease inhibitor or competitive substrate to tune the assay stringency conditions. Exemplary inhibitors include ovoinhibitor, cation chelators (e.g. EDTA), serpins, suicide inhibitors, and substrate analogs (See also Rawlings et al., 2004, Biochem. J. 378: 705-716 for additional inhibitors contemplated with the methods herein. The content of this publication is hereby incorporated by reference in its entirety.) The amount of inhibitor used in the methods may be determined by the skilled artisan based on the protease system used in view of the teachings herein (e.g., from about 250 pg/□_ to about 8000 pg/L, about 500 pg/L to about 4000 pg/L, or about 1000 pg/L).

### Formation of Emulsions

Emulsions may be produced from any suitable combination of immiscible liquids to enable a suitable platform for compartmentalizing the synthetic compounds described herein. In some embodiments, the emulsion is suitable for expressing the polypeptides (e.g., within an aqueous droplet), and those expressed polypeptides having protease activity are capable of hydrolyzing a protease substrate in that droplet.

Preferably the emulsion of the present invention has water (containing the biochemical components described supra) as the phase present in the form of finely divided droplets (the disperse, internal or discontinuous phase) and a hydrophobic, immiscible liquid (an oil) as the matrix in which these droplets are suspended (the nondisperse, continuous or external phase). Such emulsions are termed water-in-oil (W/O).

The emulsion may be stabilized by addition of one or more surface-active agents (surfactants). These surfactants are termed emulsifying agents and act at the water/oil interface to prevent (or at least delay) separation of the phases. Many oils and many emulsifiers can be used for the generation of water-in-oil emulsions; a recent compilation listed over 16,000 surfactants, many of which are used as emulsifying agents (Ash and Ash, 1993, Handbook of industrial surfactants. Gower, Aldershot). Suitable oils include light white mineral oil and non-ionic surfactants (Schick, 1966, Nonionic surfactants. Marcel Dekker, New York) such as sorbitan monooleate (Span.™.80; ICI) and polyoxyethylenesorbitan monooleate (Tween™ 80; ICI).

The use of anionic surfactants may also be beneficial. Suitable surfactants include sodium cholate and sodium taurocholate. Particularly preferred is sodium deoxycholate, preferably at a concentration of 0.5% w/v, or below. Inclusion of such surfactants can in some cases increase the expression of the polynucleotides and/or the activity of the enzymes/enzyme variants. Addition of some anionic surfactants to a non-emulsified reaction mixture completely abolishes translation. During emulsification, however, the surfactant may be transferred from the aqueous phase into the interface and activity is restored. Addition of an anionic surfactant to the mixtures to be emulsified ensures that reactions proceed only after compartmentalization.

Creation of an emulsion generally requires the application of mechanical energy to force the phases together. There are a variety of ways of doing this that utilize a variety of mechanical devices, including stirrers (such as magnetic stir-bars, propeller and turbine stirrers, paddle devices and whisks), homogenizers (including rotor-stator homogenizers, high-pressure valve homogenizers and jet homogenizers), colloid mills, ultrasound and ‘membrane emulsification’ devices (Becher, 1957, Emulsions: theory and practice. Reinhold, New York; and Dickinson, 1994, Emulsions and droplet size control. Butterworth-Heine-mann, Oxford, Vol. pp. 191-257). Accordingly, in one aspect is a method of preparing an emulsion described herein, comprising (i) suspending the plurality of synthetic compounds in the aqueous phase, and (ii) mixing the suspension of (i) with an oil.

Aqueous droplets formed in water-in-oil emulsions are generally stable with little if any exchange of polynucleotides or enzymes/enzyme variants between droplets. The technology exists to create emulsions with volumes all the way up to industrial scales of thousands of liters (Becher, 1957, Emulsions: theory and practice. Reinhold, N.Y.; Sherman, 1968, Emulsion science. Academic Press, London; Lissant, 1974, Emulsions and emulsion technology. Surfactant Science New York: Marcel Dekker; and Lissant, 1984, Emulsions and emulsion technology. Surfactant Science New York: Marcel Dekker).

The preferred droplet size will vary depending upon the precise requirements of any individual selection process that is to be performed according to the present invention. In all cases, there will be an optimal balance between polynucleotide library size, the required enrichment and the required concentration of components in the individual droplets to achieve efficient expression and reactivity of the enzymes/enzyme variants.

The processes of expression preferably occur within each individual droplet provided by the present invention. Both in vitro transcription and coupled transcription/translation become less efficient at sub-nanomolar DNA concentrations. Because of the requirement for only a limited number of DNA molecules to be present in each droplet, this therefore sets a practical upper limit on the possible droplet size. In some embodiments, the average volume of the droplets is between about 1 altoliter and about 1 nanoliter, inclusive (e.g., between about 10 altoliter and about 50 femtoliter, or about 0.5 femtoliter and about 10 femtoliter). The average diameter of the aqueous droplets typically falls within about 0.05 □m and about 100 □m, inclusive. In some embodiment, aqueous droplets in the emulsion have an average diameter between about 0.1 μm and about 50 μm, about 0.2 μm and about 25 μm, about 0.5 μm and about 10 μm, about 1 μm and about 5 μm, about 2 μm and about 4 μm, or about 3 μm and about 4 μm, inclusive. In certain embodiments, the mean volume of the droplets is less than 5.2×10¹⁶ m³ (corresponding to a spherical droplet of diameter less than 10 μm), less than 6.5×10¹⁷ m³ (corresponding to a spherical droplet of diameter less than 5 μm), less than or about 4.2×10⁻¹⁸ m³ (2 μm), or less than or about 9×10¹⁸ m³ (2.6 μm).

The effective polynucleotide concentration in the droplets may be artificially increased by various methods that will be well-known to those versed in the art. These include, for example, the addition of volume excluding chemicals such as polyethylene glycols (PEG) and a variety of gene amplification techniques, including transcription using RNA polymerases including those from bacteria such as E. coli (Roberts, 1969, Nature, 224, 1168-74; Blattner and Dahlberg, 1972, Nature New Biol. 237: 227-32; Roberts et al., 1975, Proc. Natl. Acad. Sci. USA 72: 1922-1926; Rosenberg et al., 1975, J. Biol. Chem. 250: 4755-4764), eukaryotes (Weil et al., 1979, Cell 18: 469-84; Manley et al., 1983, Methods Enzymol. 101: 568-582) and bacteriophage such as T7, T3 and SP6 (Melton et al., 1984, Nucleic Acids Res. 12: 703556); the polymerase chain reaction (peR) (Saiki et al., 1988, Science 239: 487-491); Q-beta replicase amplification (Miele et al., 1983, J. Mol. Biol. 171: 281-95; Cahill et al., 1991, Clin. Chem. 37: 1482-1485; Chetverin and Spirin, 1995, Prag. Nucleic Acid. Res. Mol. Biol. 51: 225-270; Katanaev et al., 1995, FEBS Lett. 359: 89-92); the ligase chain reaction (LCR) (Landegren et al., 1988, Science 241: 1077-1080; Barany, 1991, PCR Methods Appl. 1: 5-16); self-sustained sequence replication system (Fahy et al., 1991, PCR Methods Appl. 1, 25-33.) and strand displacement amplification (Walker et al., 1992, Nucleic Acids Res. 20: 1691-1696). Even gene amplification techniques requiring thermal cycling such as PCR and LCR could be used if the emulsions and the in vitro transcription or coupled transcription/translation systems are thermostable (for example, the coupled transcription/translation systems could be made from a thermostable organism such as Thermus aquaticus).

Increasing the effective local nucleic acid concentration enables larger droplets to be used effectively. This allows a preferred practical upper limit for most applications to the droplet volume of about 2.2×10¹⁴ m³ (corresponding to a sphere of diameter 35 μm).

The droplet size should be sufficiently large to accommodate all of the required components of the biochemical reactions that are needed to occur within the droplet, in addition to the synthetic compound. In vitro, both transcription reactions and coupled transcription/translation reactions typically employ a total nucleotide concentration of about 2 mM. For example, in order to transcribe a gene to a single short RNA molecule of 500 bases in length, this would require a minimum of 500 molecules of nucleotides per droplet (8.33×10⁻²² moles). In order to constitute a 2 mM solution, this number of molecules must be contained within a droplet of volume 4.17×10¹⁹ liters (4.17×10²² m³ which if spherical would have a diameter of 93 nm.

Furthermore, the ribosomes necessary for the translation to occur are themselves approximately 20 nm in diameter. Hence, the in some embodiments lower limit for droplets is a diameter of approximately 0.1 μm (100 nm).

The size of emulsion droplets may be varied simply by tailoring the emulsion conditions used to form the emulsion according to requirements of the selection system. The larger the droplet size, the larger is the volume that will be required to emulsify a given polynucleotide library, since the ultimately limiting factor will be the size of the droplet and thus the number of droplets possible per unit volume. In some embodiments, the emulsion comprises at least about 10², 10³, 10⁴, 10⁵, 10⁶, 10⁷, 10⁸, 10⁹, 10¹⁰, 10¹¹, 10¹², or 10¹⁵ droplets/mL of emulsion.

Depending on the complexity and size of the library to be screened, it may be beneficial to form an emulsion such that in general 1 or less than 1 synthetic compound is included in each droplet of the emulsion. The number of synthetic compounds per droplet is governed by the Poisson distribution. Accordingly, if conditions are adjusted so that there are, on average, 0.1 synthetic compound per droplet, then, in practice, approximately: 90% of droplets will contain no synthetic compound, 9% of droplets will contain 1 synthetic compound, and 1% of droplets will contain 2 or more synthetic compounds. In practice, average values of about 0.1 to about 0.5, more preferably about 0.3, synthetic compounds per droplet provide emulsions that contain a sufficiently high percentage of droplets having 1 synthetic compound per droplet, with a sufficiently low percentage of droplets having 2 or more synthetic compounds per droplet. This approach will generally provide the greatest power of resolution. Where the library is larger and/or more complex, however, this may be less practical; it may be preferable to include several synthetic compound together and rely on repeated application of the method of the invention to achieve sorting of the desired activity. In some embodiments, no more than 70%, 60%, 50%, 40%, 30%, 20%, 15%, 10% or 5% of the aqueous droplets of the water-in-oil emulsion comprise more than one synthetic compound

Theoretical studies indicate that the larger the number of polynucleotide mutants created the more likely it is that a corresponding encoded polypeptide will be created with the properties desired (See, e.g., Perelson and Oster, 1979 for a description of how this applies to repertoires of antibodies). Recently it has also been confirmed practically that larger phage-antibody repertoires do indeed give rise to more antibodies with better binding affinities than smaller repertoires (Griffiths et al., 1994). To ensure that rare variants are generated and thus are capable of being selected, a large library size is generally desirable.

Using the present system, at an aqueous droplet diameter of 2.6 μm, a repertoire size of at least 10¹¹ can readily be sorted using 1 ml aqueous phase in a 20 ml emulsion.

### Expression, Separation and Further Processing

The emulsion is maintained for a sufficient time under conditions suitable for expression of the polypeptides. The active proteases act to hydrolyze the protease substrate attached to the polynucleotides in that droplet. By attenuating the expression conditions using the teachings described herein, the gene coding sequences for those polypeptides with enhanced protease activity can be distinguished from those having less activity.

In some embodiments, expression occurs by incubating the emulsion at about 25° C. to about 60° C. (e.g., about 25° C. to about 50° C., about 30° C. to about 40° C.) for about 1 hour to about 24 hours (e.g., about 1 hour to about 12 hours, about 1 hour to about 5 hours, or about 1 hour to about 2 hours).

In some embodiments, the aqueous phase is separated from the oil phase (e.g., prior to step (iv)) by any suitable technique, such as, for example chemically-induced coalescence and/or centrifugation.

The hydrolyzed synthetic compounds may be separated from the non-hydrolyzed synthetic compounds using any of a number of conventional techniques. For example, separation of non-hydrolyzed from hydrolyzed substrate may be accomplished, e.g., by using C18 magnetic beads (e.g. Dynabeads® RPC 18, Thermo Fisher Scientific, Inc.). Magnetic silica beads coated with C4, C8, and C18 alkyl groups are routinely used to separate hydrophobic species (e.g., non-hydrolyzed synthetic compounds having fatty acid chains intact). Separation my also occur by removing non-hydrolyzed compounds through binding to silica or anion exchange, or charge switch media, as known in the art. Further, as described supra, separation can further be aided when the synthetic compound comprises a selectable marker, where, e.g., antibodies, lectin, or streptavidin can bind to the marker and remove non-hydrolyzed compounds by affinity capture.

In some embodiments, the recovered hydrolyzed and/or non-hydrolyzed synthetic compounds results in the compounds being substantially pure. With respect to hydrolyzed synthetic compounds, “substantially pure” intends a recovered preparation of hydrolyzed synthetic compounds that contains no more than 15% impurity, wherein impurity intends non-hydrolyzed synthetic compounds. With respect to non-hydrolyzed synthetic compounds, “substantially pure” intends a recovered preparation of non-hydrolyzed synthetic compounds that contains no more than 15% impurity, wherein impurity intends hydrolyzed synthetic compounds. In some variations, substantially pure hydrolyzed synthetic compounds or non-hydrolyzed synthetic compounds may contain no more than 10% impurity, or no more than 5% impurity, or no more than 3% impurity, or no more than 1% impurity, or no more than 0.5% impurity.

The collection of separated synthetic compounds (hydrolyzed and/or non-hydrolyzed) may be further analyzed. For example, after each round of selection, the enrichment of the pool of polynucleotides for those encoding a protease of interest can be analyzed, e.g., by non-compartmentalized sequencing reactions known in the art. In one embodiment, the method further comprises analyzing the polynucleotide sequence (e.g., via sequencing) of one or more of the separated synthetic compounds of step (iv), such as one or more of the hydrolyzed synthetic compounds, and/or one or more of the non-hydrolyzed compounds.

The selected pool can be amplified and/or cloned into a suitable expression vector for propagation and/or expression, as described below, using techniques known in the art. In one embodiment, the method further comprises amplifying one or more polynucleotides of the one or more hydrolyzed synthetic compounds of step (iv). In another embodiment, the method further comprises amplifying one or more polynucleotides of the one or more non-hydrolyzed synthetic compounds of step (iv).

The polynucleotides of the separated synthetic compounds may also be subjected to subsequent, possibly more stringent rounds of sorting in iteratively repeated steps, reapplying the method of the invention either in its entirety or in selected steps only. By tailoring the conditions appropriately, synthetic compounds encoding proteases having a better optimized activity may be generated after each round of selection. Accordingly, in some embodiments, the method is reiterated wherein the polynucleotides of the separated synthetic compounds (e.g., the amplified polynucleotides from the hydrolyzed synthetic compounds) are used in a new plurality of synthetic compounds as described in step (i), and steps (i)-(iv) are repeated with said new plurality of synthetic compounds. If desired, further genetic variation can be introduced into the polynucleotides prior to repeating the method, using, e.g., error-prone polymerase chain reaction (PCR) and/or other techniques described supra. Accordingly, in one embodiment, the method further comprising introducing an alteration to (e.g., via mutagenizing) one or more polynucleotides of the separated synthetic compounds of step (iv).

### Nucleic Acid Constructs and Expression Vectors

In some embodiments, the methods described herein further comprise cloning one or more polynucleotides of the separated synthetic compounds from step (iv) into a nucleic acid construct or expression vector. RNA and/or recombinant protein can be produced from the individual clones for further purification and assay (as described below). Recombinant selected using the methods of the invention can be employed for any application for which the native enzyme is employed. Thus, in some embodiments, the methods further comprise expressing one or more of polynucleotides from the separated synthetic compounds of step (iv) (e.g., expressing a polynucleotide of a hydrolyzed synthetic compound to produce a polypeptide with protease activity).

The nucleic acid constructs comprise a polynucleotide encoding a polypeptide or variant described herein operably linked to one or more control sequences that direct the expression of the coding sequence in a suitable host cell under conditions compatible with the control sequences.

The polynucleotide may be manipulated in a variety of ways to provide for expression of a polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

In some embodiments, the nucleic acid constructs and expression vectors use a bacterial expression system (e.g., a Bacillus expression system).

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis crylliA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol 3-hydroxypropionate dehydrogenase/glyceraldehyde-3-phosphate 3-hydroxypropionate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator sequence is operably linked to the 3′-terminus of the polynucleotide encoding the variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate 3-hydroxypropionate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryllIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader sequence is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol 3-hydroxypropionate dehydrogenase/glyceraldehyde-3-phosphate 3-hydroxypropionate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus nigerglucoamylase, Aspergillus oryzae TAKA amylase, Humicola insolens cellulase, Humicola insolens endoglucanase V, Thermomyces lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked with the regulatory sequence.

Recombinant expression vectors comprise a polynucleotide encoding a polypeptide or variant described herein, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

In some embodiments, the methods described herein further comprise transforming one or more polynucleotides of the separated synthetic compounds from step (iv) (e.g., a nucleic acid construct or expression vector comprising the polynucleotide) into a recombinant host cell. A construct or vector comprising a polynucleotide is introduced into a host cell so that the construct or vector is maintained as a chromosomal integrant or as a self-replicating extra-chromosomal vector. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a protease of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Thermomyces lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J.N. and Simon, M.I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

In some embodiments, the methods described herein further comprise cultivating a recombinant host cell described supra under conditions suitable for expression of the protease, and optionally recovering the protease.

The host cells are cultivated in a nutrient medium suitable for production of the protease using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The protease may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the protease.

The protease may be recovered using methods known in the art. For example, the protease may be recovered from the nutrient medium by conventional procedures including, but not limited to, collection, centrifugation, filtration, extraction, spray-drying, evaporation, or precipitation. In one aspect, a whole fermentation broth comprising the protease is recovered.

The protease may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the protease is not recovered, but rather a host cell of the present invention expressing the protease is used as a source of the polypeptide.

The present invention may be further described by the following numbered paragraphs:

Paragraph [1]: A method of selecting for a polypeptide having protease activity, the method comprising:

(i) suspending a plurality of synthetic compounds in an aqueous phase, wherein the synthetic compounds individually comprise:
(a) a polynucleotide encoding for a polypeptide;
(b) a protease substrate linked to said polynucleotide; and
(c) a selectable marker linked to said polynucleotide; wherein the aqueous phase comprises components for expression of the polypeptide;

(ii) forming a water-in-oil emulsion with the aqueous phase, wherein the synthetic compounds are compartmentalized in aqueous droplets of the emulsion;

(iii) expressing the polypeptides within the aqueous droplets of the emulsion, wherein a polypeptide with protease activity in an aqueous droplet hydrolyzes the protease substrate in that droplet; and

(iv) separating the synthetic compounds to recover synthetic compounds comprising the protease substrate and/or synthetic compounds wherein the protease substrate has been hydrolyzed.

Paragraph [2]: The method of paragraph [1], wherein the polypeptide comprises a propeptide.
Paragraph [3]: The method of paragraph [1] or [2], wherein the plurality of synthetic compounds comprises at least about 10⁶ different synthetic compounds (e.g., at least about 10¹⁰, 10¹², or 10¹⁴ different synthetic compounds).
Paragraph [4]: The method of any one of the preceding paragraphs, wherein no more than 20% of the aqueous droplets of the water-in-oil emulsion comprise more than one synthetic compound.
Paragraph [5]: The method of any one of the preceding paragraphs, wherein each synthetic compound comprises only one copy of one polynucleotide.
Paragraph [6]: The method of any one of the preceding paragraphs, wherein the emulsion comprises at least about 10⁶ aqueous droplets/mL of emulsion (e.g., at least about 10⁹, 10¹², or 10¹⁵ aqueous droplets/mL of emulsion).
Paragraph [7]: The method of any one of the preceding paragraphs, wherein the aqueous droplets in the emulsion have an average diameter between about 0.05 μm and about 100 μm, inclusive (e.g., between about 0.1 μm and about 50 μm, about 0.2 μm and about 25 μm, about 0.5 μm and about 10 μm, or about 1 μm and about 5 μm, inclusive).
Paragraph [8]: The method of any one of the preceding paragraphs, wherein the aqueous droplets in the emulsion have an average volume of between about 1 altoliter and about 1 nanoliter, inclusive (e.g., between about 10 altoliter and about 50 femtoliter, or about 0.5 femtoliter and about 10 femtoliter).
Paragraph [9]: The method of any one of the preceding paragraphs, wherein the polynucleotide encoding for a polypeptide is linked to the protease substrate with a substituted thiol (e.g., thioether), substituted amino (e.g., amido), or triazole moiety.
Paragraph [10]: The method of any one of the preceding paragraphs, wherein the selectable marker is linked to the polynucleotide in a distal position relative to the protease substrate.
Paragraph [11]: The method of any one of the preceding paragraphs, wherein the selectable marker is linked to polynucleotide with a substituted thiol (e.g., thioether), substituted amino (e.g., amido), or triazole moiety.
Paragraph [12]: The method of any one of the preceding paragraphs, wherein the selectable marker is an affinity tag.
Paragraph [13]: The method of paragraph [12], wherein the affinity tag comprises biotin.
Paragraph [14]: The method of paragraph [13], wherein the hydrolyzed synthetic compounds of step (iv) are separated from the non-hydrolyzed synthetic compounds with streptavidin (e.g., streptavidin coated microspheres).
Paragraph [15]: The method of any one of the preceding paragraphs, wherein the synthetic compounds individually comprise a solid phase.
Paragraph [16]: The method of paragraph [15], wherein the solid phase is linked to said protease substrate, and wherein hydrolysis of the protease substrate releases the solid phase from the synthetic compound.
Paragraph [17]: The method of paragraph [15] or [16], wherein the solid phase is a microbead or particle.
Paragraph [18]: The method of paragraph [17], wherein the solid phase is a hydrophobic microbead.
Paragraph [19]: The method of any one of paragraphs [15]-[18], wherein the solid phase is a gold nanoparticle.
Paragraph [20]: The method of any one of the preceding paragraphs, wherein the aqueous phase further comprises a protease inhibitor and/or competitive substrate.
Paragraph [21]: The method of any one of the preceding paragraphs, comprising separating the aqueous phase from the oil phase (e.g., via chemically-induced coalescence and/or centrifugation) prior to step (iv).
Paragraph [22]: The method of any one of the preceding paragraphs, wherein the recovered synthetic compounds comprising the protease substrate and/or synthetic compounds wherein the protease substrate has been hydrolyzed are substantially pure.
Paragraph [23]: The method of any one of the preceding paragraphs, further comprising analyzing the polynucleotide sequence (e.g., via sequencing) of one or more of the separated synthetic compounds of step (iv).
Paragraph [24]: The method of any one of the preceding paragraphs, further comprising amplifying one or more polynucleotides of the synthetic compounds of step (iv) wherein the protease substrate has been hydrolyzed.
Paragraph [25]: The method of any one of the preceding paragraphs, further comprising amplifying one or more polynucleotides of the synthetic compounds of step (iv) comprising the protease substrate.
Paragraph [26]: The method of paragraph [24] or [25], wherein the amplified one or more polynucleotides are used in a new plurality of synthetic compounds as described in step (i), and steps (i)-(iv) are repeated with said new plurality of synthetic compounds.
Paragraph [27]: The method of any one of the preceding paragraphs, further comprising introducing an alteration to (e.g., mutagenizing) one or more polynucleotides of the separated synthetic compounds of step (iv).
Paragraph [28]: The method of paragraph [27], wherein the one or more altered polynucleotides are used in a new plurality of synthetic compounds as described in step (i), and steps (i)-(iv) are repeated with said new plurality of synthetic compounds.
Paragraph [29]: The method of any one of the preceding paragraphs, further comprising expressing one or more of polynucleotides from the separated synthetic compounds of step (iv) (e.g., expressing a polynucleotide of a synthetic compound wherein the protease substrate has been hydrolyzed, thereby producing a polypeptide with protease activity).
Paragraph [30]: The method of any one of the preceding paragraphs, further comprising cloning one or more polynucleotides of the separated synthetic compounds from step (iv) into an expression vector.
Paragraph [31]: The method of paragraph [30], further comprising transforming said expression vector into a recombinant host cell.
Paragraph [32]: The method of paragraph [31], further comprising cultivating the recombinant host cell under conditions suitable for expression of the polypeptide, and optionally recovering the polypeptide.
Paragraph [33]: A synthetic compound comprising:

(a) a polynucleotide encoding for a polypeptide;

(b) a protease substrate linked to said polynucleotide; and

(c) a selectable marker linked to said polynucleotide.

Paragraph [34]: The synthetic compound of paragraph [33], wherein the polypeptide comprises a propeptide.
Paragraph [35]: The synthetic compound of paragraph [33] or [34], which comprises only one copy of one polynucleotide.
Paragraph [36]: The synthetic compound of any one of paragraphs [33]-[35], wherein the polynucleotide encoding for a polypeptide is linked to the protease substrate with a substituted thiol (e.g., thioether), substituted amino (e.g., amido), or triazole moiety.
Paragraph [37]: The synthetic compound of any one of paragraphs [33]-[36], wherein the selectable marker is linked to the polynucleotide in a distal position relative to the protease substrate.
Paragraph [38]: The synthetic compound of any one of paragraphs [33]-[37], wherein the mature form of the polypeptide has protease activity.
Paragraph [39]: The synthetic compound of any one of paragraphs [33]-[38], wherein the polypeptide is a protease variant.
Paragraph [40]: The synthetic compound of any one of paragraphs [33]-[39], wherein the selectable marker is linked to polynucleotide with a substituted thiol (e.g., thioether), substituted amino (e.g., amido), or triazole moiety.
Paragraph [41]: The synthetic compound of any one paragraphs [33]-[40], wherein the selectable marker is an affinity tag.
Paragraph [42]: The synthetic compound of paragraph [41], wherein the affinity tag comprises biotin.
Paragraph [43]: The synthetic compound of any one of paragraphs [33]-[42], further comprising a solid phase.
Paragraph [44]: The synthetic compound of paragraph [43], wherein the solid phase is linked to said protease substrate.
Paragraph [45]: The synthetic compound of paragraph [43] or [44], wherein the solid phase is a microbead or particle.
Paragraph [46]: The synthetic compound of paragraph [45], wherein the solid phase is a hydrophobic microbead.
Paragraph [47]: The synthetic compound of any one of paragraphs [43]-[45], wherein the solid phase is a gold nanoparticle.
Paragraph [48]: The synthetic compound of any one of paragraphs [33]-[47], which is capable of being hydrolyzed when contacted with a polypeptide having protease activity.
Paragraph [49]: A method of making the synthetic compound of any one of paragraphs [33]-[48], comprising:

(i) linking a protease substrate to a polynucleotide encoding for a polypeptide;

(ii) linking a selectable marker to the polynucleotide encoding for a polypeptide; and

(ii) recovering the synthetic compound.

Paragraph [50]: The method of paragraph [49], further comprising linking the protease substrate to a solid phase.
Paragraph [51]: A polynucleotide library comprising a plurality of different synthetic compounds according to any one of paragraphs [33]-[48].
Paragraph [52]: The polynucleotide library of paragraph [51], wherein the plurality of synthetic compounds comprises at least about 10⁶ different synthetic compounds (e.g., at least about 10¹⁰, 10¹², or 10¹⁴ different synthetic compounds).
Paragraph [53]: A water-in-oil emulsion comprising the polynucleotide library of paragraph [51] or [52], wherein the synthetic compounds are compartmentalized in aqueous droplets of the emulsion.
Paragraph [54]: The emulsion of paragraph [53], wherein no more than 20% of the aqueous droplets of the water-in-oil emulsion comprises more than one synthetic compound.
Paragraph [55]: The emulsion of paragraph [53] or [54], further comprising components for expression of the polypeptide in the aqueous droplets.
Paragraph [56]: The emulsion of any one of paragraphs [53]-[55], further comprising an emulsifying agent.
Paragraph [57]: The emulsion of any one of paragraphs [53]-[56], comprising at least about 10⁶ aqueous droplets/mL of emulsion (e.g., at least about 10⁹, 10¹², or 10¹⁵ aqueous droplets/mL of emulsion).
Paragraph [58]: The emulsion of any one of paragraphs [53]-[57], wherein the aqueous droplets have an average diameter between about 0.05 μm and about 100 μm, inclusive (e.g., between about 0.1 μm and about 50 μm, about 0.2 μm and about 25 μm, about 0.5 μm and about 10 μm, or about 1 μm and about 5 μm, inclusive).
Paragraph [59]: The emulsion of any one of paragraphs [53]-[58], wherein the aqueous droplets have an average volume of between about 1 altoliter and about 1 nanoliter, inclusive (e.g., between about 10 altoliter and about 50 femtoliter, or about 0.5 femtoliter and about 10 femtoliter).
Paragraph [60]: The emulsion of any one of paragraphs [53]-[59], wherein the emulsion is suitable for expressing the polypeptides within the aqueous droplets.
Paragraph [61]: The emulsion of any of one of paragraphs [53]-[60], wherein the expressed polypeptides having protease activity are capable of hydrolyzing one or more synthetic compounds in that droplet.
Paragraph [62]: A method of making the emulsion of any one of paragraphs [53]-[61], comprising:

(i) suspending the plurality of synthetic compounds in the aqueous phase; and

(ii) mixing the suspension of (i) with an oil.

### EXAMPLES

The following examples are provided by way of illustration and are not intended to be limiting of the invention.

Chemicals used as buffers and substrates were commercial products of at least reagent grade.

### Example 1: Preparing DNA for Bioconjugation

Two separate PCRs were performed with either template A (SEQ ID NO: 1, containing the coding sequence of SEQ ID NO: 2, and encodes for the polypeptide of SEQ ID NO: 3) or template B (SEQ ID NO: 4, containing the coding sequence of SEQ ID NO: 5, and encodes for the polypeptide of SEQ ID NO: 6) as follows: A 50 μL aq. reaction was assembled containing 0.5 μM biotin-TEG-modified Primer A (5′-BiotinTEG/SEQ ID NO: 7; 5′-CGG TTT CTT GGC CTC CAT ATA C-3′), 0.5 μM TCO-modified Primer B (SEQ ID NO: 8; 5′-AAG TCA GTA CGT GTG CGC TTA TAG-3′), 1 ng of template A or template B, and 25 μL Q5® High-Fidelity 2× Master Mix (New England Biolabs, Ipswich, Mass. USA). The reaction was activated at 98° C. for 30 s and then thermal cycled 24 times (98° C. for 5 s, 65° C. for 15 s, 72° C. for 30 s) followed by a final extension at 72° C. for 2 m. The resulting amplicon A or amplicon B were purified from residual PCR components using the High Prep PCR system (MAGBIO GENOMICS, Gaithersburg, Md. USA) according to the manufacturer's protocol.

### Example 2: Coating Protein Substrates onto Microspheres

### Protein Substrate on Tosylactivated Beads

α-casein was covalently attached to the surface of magnetic microspheres as follows: Dynabeads MyOne™ Tosylactivated (Thermo Scientific, Inc.) 100 mg/mL stock suspension was thoroughly mixed by vortexing. 500 μL of the suspension was transferred to a microfuge tube and placed on a magnet stand (Promega MagneSphere® magnetic separation stand) for ≥2 m, allowing the beads to migrate to the side of the tube and the liquid to be cleared of suspended beads. The supernatant was removed by pipetting and discarded, leaving the microspheres in the microfuge tube. The microspheres were washed 3× by repeated addition and removal of 1 mL of Coating Buffer (0.1 M Sodium Borate (H₃BO₃+NaOH), pH 9.5) using the same procedure used to remove the supernatant. The washed microsphere pellet was resuspended in 805 μL of Coating Buffer. 30 μL of 80 mg/mL α-casein was added to the resuspended microspheres and mixed by vortexing. 415 μL of 3 M ammonium sulphate was added to the suspension and the microfuge tube was incubated with slow tilt rotation for 16-24 h at 37° C. After incubation the supernatant was removed using the magnetic stand as before.

The surface of the microspheres was then passivated by resuspending the microspheres in 1.25 mL of Blocking Buffer (1× PBS, with 0.5% (w/v) Glycine and 0.05% Tween-20) followed by incubation for 16-24 h at 37° C. with slow tilt rotation. The passivated microspheres were washed 3× by repeated addition and removal of 1 mL of Washing Buffer (1× PBS with 0.1% Glycine and 0.05% Tween 20) using the same procedure used to remove the supernatant. Washed microspheres were resuspended to 500 μL with the addition of ˜300 μL of Storage Buffer (1× PBS, with 0.1% Glycine, 0.05% Tween-20 and 0.02% Sodium Azide). The microsphere suspension was sonicated to disperse the microspheres before storage.

Similarly, other protein substrates such as bovine serum albumin or hemoglobin can be coated onto microspheres using the same procedure.

### Protein Substrate on Carboxylated Beads

Bovine Serum Albumin (BSA, Sigma-Aldrich #A3294-50G) was covalently attached to the surface of magnetic microspheres as follows: BSA was dissolved at 80 mg/mL in 15 mM MES (2-(N-morpholino)ethanesulfonic acid), pH 6 with overnight rotation at room temperature. Dynabeads MyOne™ Carboxylic Acid (Thermo Scientific, Inc.) 10 mg/mL stock suspension was thoroughly mixed by vortexing. 1 mL of the suspension was transferred to a siliconized microfuge tube (Ambion™ Nonstick, RNase-free Microfuge Tubes, 2.0mL #AM12475) and placed on a magnet stand (Promega MagneSphere® magnetic separation stand) for ≥2 m, allowing the beads to migrate to the side of the tube and the liquid to be cleared of suspended beads. The supernatant was removed by pipetting and discarded, leaving the microspheres in the microfuge tube. The microfuge tube was removed from the magnet and the microspheres were washed 2× by repeated addition and removal of 1 mL of 15 mM MES, pH 6 using the same procedure used to remove the supernatant. The washed microsphere pellet was resuspended in 100 μL of 15 mM MES buffer, pH 6. 100 μL of freshly-dissolved EDC (1-ethyl-3-(3-dimethylaminopropyl)carbodiimide hydrochloride) at 10 mg/mL in cold PCR-grade water was added to the microsphere suspension and incubated with rotation for 30 m at room temperature. The supernatant was removed by placing the microfuge tube on a magnet stand for ≥2 m as before. The BSA solution at 80 mg/mL was diluted 100-fold in 15 mM MES, pH 6 and 500 μL of the dilution was added to the EDC-activated microspheres. BSA was bound to the microsphere surface with 16-24 h rotation at room temperature.

The supernatant was removed by placing the microfuge tube on a magnet stand for ≥2 m as before. The microspheres were washed 2× by repeated addition of 1 mL of 1× PBS, 0.1% Tween 20, pH 7.4 with rotation at room temperature for 10 m, and buffer removal using the same procedure used to remove the supernatant. After washing, the supernatant was removed by placing the microfuge tube on a magnet stand for ≥2 m as before. The microspheres were resuspended in 200-500 μL of storage buffer (1× PBS, 0.1% Tween 20, pH 7.4). Prior to DNA conjugation (Example 3), the microspheres are dispersed by sonication for 1 h.

### Example 3: Linking Substrate to DNA

Biotin- and TCO-modified DNA amplicons from Example 1 were conjugated to the protein-coated microspheres as follows: α-casein-coated magnetic microspheres were resuspended at 20 mg/mL in 100 mM phosphate, 0.05% Tween-20 (pH 8) buffer using the magnetic stand as in Example 2 to remove the Storage Buffer. Resuspended microspheres were sonicated for 30 m to disperse the microspheres. Microspheres were washed 6× in 600 μl of PB/T Buffer (100 mM phosphate, 0.05% Tween-20, pH 8) using the magnetic stand as in Example 2. Washed microspheres were resuspended at 100 mg/mL in PB/T Buffer. 0.5 μmol NHS-Tetrazine was combined with 33.3 μL of dry DMF, resulting in a 15 mM NHS-Tetrazine solution. 45 μL of 100 mg/mL washed microspheres was combined with 4 μL of 15 mM NHS-Tetrazine and 11 μL of PB/T buffer. The NHS-Tetrazine was reacted with the α-casein on the microspheres at room temperature for 3 h with shaking at 800 rpm. The tetrazine-activated microspheres were washed 6× in PB/T buffer as before and resuspended at 100 mg/mL in PB/T buffer. To reduce non-specific DNA binding, 20 μL of SuperBlock Buffer was added and incubated for 1 h at room temperature with slow tilt rotation.

After incubation, the microsphere suspension was placed on the magnetic stand and 20 μL of supernatant removed to return the microsphere concentration to 100 mg/mL. The microspheres were resuspended and 10 μL was combined with 4.9 μL of either amplicon A or amplicon B (Example 1) at 2 ng/μL and 4.1 μL of SuperBlock buffer. The reactions were incubated 16 h at 50° C. with continual rotation. After incubation, 160 μL of PB/T was added and the entire volume processed in a Kingfisher (Thermo Scientific) automated magnetic particle processing instrument to wash the microspheres 6× in 180 μL of 1× PBS. The washed microspheres were resuspended in 360 μL of 1× PBS. Microsphere concentration was measured by absorbance at 400 nm in a BioTek plate reader and normalized to 5 mg/mL in 1× PBS.

### Example 4: Emulsion Formation and Polypeptide Expression

The DNA-microsphere conjugates of Example 3 were emulsified using the following procedure: A 143.9 μL aq. in vitro transcription/translation (IVTT) reaction was assembled on ice using the PURExpress® In Vitro Protein Synthesis Kit (New England Biolabs, Ipswich, Mass. USA). The IVTT reaction contained 67 μL PURExpress® Tube A, 6.7 μL PURExpress® Disulfide Bond Enhancer 2, 3.4 μL Murine RNase Inhibitor (#M0314), 48.6 μL PURExpress® Tube B, and 1 μg of random blocking DNA. The DNA-microsphere conjugates from Example 3 were washed 3× with 1× PB/T as before and resuspended in 50 mM HEPES at a concentration of 5 mg/mL. Microspheres conjugated to amplicon A and amplicon B were pooled such that approximately 2% were derived from SEQ ID NO: 1 (encoding for wild-type Savinase) and 98% were derived from SEQ ID NO: 4 (encoding for the catalytically inactive Savinase). The cold 143.9 μL IVTT reaction was combined with 23.6 μL of the pooled DNA-microsphere conjugates and placed in a 2 mL round-bottom tube (Eppendorf AG, Hamburg Germany) containing a 3 mm tungsten carbide bead (Qiagen, Venlo, Limburg) and 335 μL of room temperature 3M Novec HFE-7500, 2% Pico-Surf 1 (The Dolomite Centre Ltd., Royston, UK).

The tube was agitated in a TissueLyser (Qiagen, Venlo Limburg) at 15 Hz for 10 s followed by 17 Hz for 60 s. Emulsions were incubated at 30° C. for 1 to 4 h to allow polypeptide expression and protein hydrolysis. For some tests, the emulsion temperature was raised to 40° C. for additional 1 to 36 hr to investigate the effects of higher temperature on protein hydrolysis.

After expression/hydrolysis, the aqueous fraction was recovered as follows: 10 μL of 20 mM PMSF was added to each emulsion to prevent further protein hydrolysis once the emulsion was broken. 500 μL of Pico-Break 1 (The Dolomite Centre Ltd., Royston, UK) was added to the emulsion and then inverted until a homogenous suspension was achieved. The tubes were spun briefly to remove suspension from the tube cap and placed on a magnet stand for 30 s. Phase Lock Gel (PLG) Heavy, 2 mL (5Prime, Fisher FP2302830) tubes were prepared by centrifugation at 14,000 ×g in a microcentrifuge for 25 s to pellet PLG. The entire suspension was transferred to the prepared PLG tubes, leaving as many microspheres behind as possible. The tubes were centrifuge at 14,000 ×g for 5 m to separate the phases. The top aqueous fraction was carefully removed by pipetting and transferred to a clean tube. The PLG tubes were back-extracted with 30 μL of IDTE (10 mM Tris, pH 8.0, 0.1 mM EDTA), centrifugation at 14,000 ×g for 30 s, and combined with the top aqueous fraction. The total aqueous fraction volume was raised to 160 μL with the addition of IDTE. Residual microspheres were removed by placing tubes on a magnetic stand for 30 s and transferring the entire 160 μL supernatant into a clean tube.

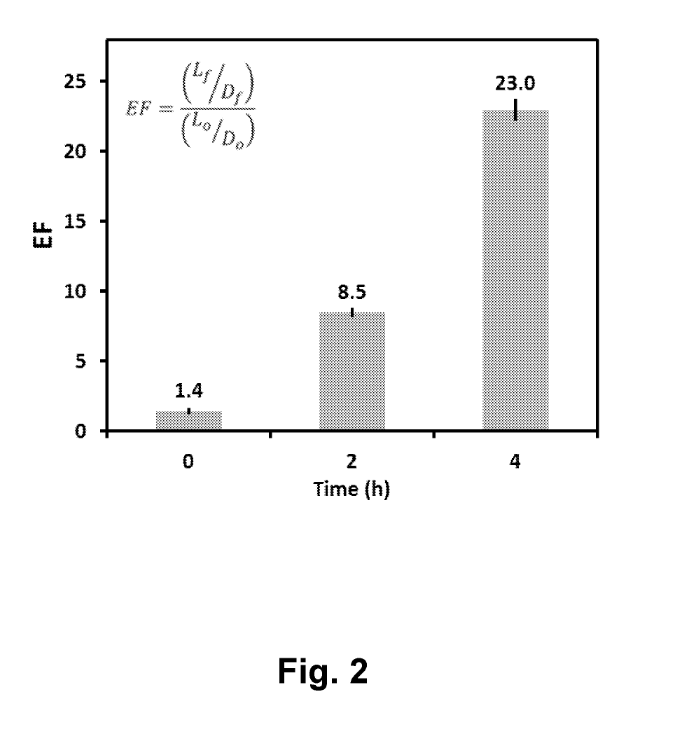

Measurement of enrichment of amplicon A compared to amplicon B was performed by droplet digital PCR (ddPCR) as follows: 22 μL aq. reactions were assembled containing 900 nM Primer 1 (SEQ ID NO: 27; 5′-GTTC AACA TATG CCAG CTT-3′) and 900 nM Primer 2 (SEQ ID NO: 28; 5′-CGCAC CTGCA ACATG A-3′), 250 nM Probe 1 (active) (5′-/5HEX/ACGG TACA TCGA TGGC (SEQ ID NO: 29)/3IABkFQ/-3′) and 250 nM Probe 2 (inactive) (5′-/56-FAM/ACGG TACA GCAA TGGC (SEQ ID NO: 29)/3IABkFQ/-3′), 2.2 μL of the recovered DNA diluted to approximately 40,000 molecules/μL, and 11 μL 2× ddPCR Supermix for Probes (no dUTP) Control (#720001476 Bio-Rad, Hercules, Calif. USA). Droplets were generated by using an Automated Droplet Generator (#1864101 Bio-Rad, Hercules, Calif. USA) according to the manufacturer's protocols. Droplets were thermal cycled 40 times (95° C. for 30 s, 52° C. for 1 m), and then read on a QX200™ Droplet Reader (#1864003 Bio-Rad, Hercules, Calif. USA) to determine active and inactive variant ratios. Enrichment in FIG. 2 is presented as the Enrichment Factor (EF), which is the quotient of the final ratio of active to inactive alleles (L_{final} and D_{final}, respectively) and the starting ratio of active to inactive alleles (L₀ and D₀, respectively).

### Example 5: Recovery of Released DNA Using Affinity Capture

Biotinylated DNA molecules encoding enzymes with activity toward the substrate are released from the DNA-microsphere conjugates and captured on streptavidin-coated beads as follows: A Kingfisher automated magnetic particle processor (Thermo Scientific, Inc.) was used to perform washing and DNA binding steps. Dynabeads® MyOne™ Streptavidin C1 magnetic beads (Thermo Fisher Scientific, Inc.) were washed once in a 200-μL volume of 1× Bind & Wash Buffer (5 mM Tris-HCl pH 7.5, 0.5 mM EDTA, 1 M NaCl, 0.01% Tween-20). The 160 μL supernatant from Example 4 was combined with 40 μL of 5× Bind & Wash Buffer to make a 200 μL solution in 1× Bind & Wash Buffer. The washed magnetic beads were then added to the solution and incubated at room temperature for 30 m with intermittent agitation to keep the magnetic beads in suspension. The magnetic beads containing captured DNA molecules were washed 2× in a 200-μL volume of 1× Bind & Wash Buffer and then once in a 200 μL volume of 0.1× Bind & Wash Buffer. The beads were resuspended in 200 μL of IDTE, 0.01% Tween-20 and the volume transferred to a clean 1.5 mL DNA lo-bind microfuge tube. The tube was manually placed on a magnetic stand for 60 s and the supernatant was carefully removed and discarded from the tube without disturbing the bead pellet. The beads were resuspended in 18 μL IDTE, 0.01% Tween-20.

The biotinylated, enriched amplicon pool bound to streptavidin coated beads (supra) was PCR amplified as follows: A 50 μL aq. reaction was assembled containing 0.5 μM Primer A (SEQ ID NO: 7), 0.5 μM Primer B (SEQ ID NO: 9; 5′-GTC AGT ACG TGT GCG CTT ATA G-3′), 10 □_ of the bead solution (supra) and 25 μL Q5® High-Fidelity 2× Master Mix (New England Biolabs, Ipswich, Mass. USA). The reaction was activated at 98° C. for 30 s and then thermal cycled 10 times (98° C. for 5 s, 65° C. for 15 s, 72° C. for 30 s) followed by a final extension at 72° C. for 2 m. The resulting DNA pool was purified from residual PCR components using the High Prep PCR system (MAGBIO GENOMICS, Gaithersburg, Md. USA) according to the manufacturer's protocol.

The purified DNA pool was then PCR amplified as follows: A 50 μL aq. reaction was assembled containing 0.5 μM biotin-TEG-modified Primer A (5′-BiotinTEG/SEQ ID NO: 7), 0.5 μM TCO-modified Primer B (SEQ ID NO: 8), 10 uL of purified amplicon from the step above, and 25 μL Q5® High-Fidelity 2× Master Mix (New England Biolabs, Ipswich, Mass. USA). The reaction was activated at 98° C. for 30 s and then thermal cycled 26 times (98° C. for 5 s, 65° C. for 15 s, 72° C. for 30 s) followed by a final extension at 72° C. for 2 m. The resulting amplicon pool was purified from residual PCR components using the High Prep PCR system (MAGBIO GENOMICS, Gaithersburg, Md. USA) according to the manufacturer's protocol. PCR pool was band purified for the size of interest (1200 bp) using the Pippin HT (Sage Science, Inc. Beverly, Mass., USA) 1.5% agarose cassette, 15 C marker, and a broad range cut.

The amplified biotin and -TCO modified enriched pool (supra) was made single-stranded using the following procedure: A Kingfisher automated magnetic particle processor (Thermo Scientific, Inc.) was used to preform washing, binding and DNA melting steps. Dynabeads® MyOne™ Streptavidin C1 magnetic beads (Thermo Fisher Scientific, Inc.) were washed once in 200 μL volume of 1× Bind & Wash Buffer (5 mM Tris-HCl pH 7.5, 0.5 mM EDTA, 1 M NaCl, 0.01% Tween-20). 0.5 μg of DNA from the Pippin HT elution was bound to the MyOne™ C1 beads in the presence of 1× Bind and Wash Buffer. The magnetic beads containing captured DNA molecules were washed 3× in 200 μL 1× Bind & Wash solution, 1× in 0.1× Bind & Wash solution, then in IDTE (10 mM Tris, pH 8.0, 0.1 mM EDTA). The bead bound DNA was then incubated in 75 μL of 150 mM NaOH, for 30 min with gentle agitation, leaving the biotin-tagged strand bound to the streptavidin beads and the TCO-labeled strand in free solution. The beads, and bead bound DNA were then removed from the NaOH solution leaving just the free solution, TCO-tagged strand of each amplicon in the enriched pool. Each 75 μL NaOH, ssDNA solution was neutralized by adding 1.1 μL 1 M Tris-HCl pH 8.0 and 6.25 μL 1.25 M acetic acid. Samples were then buffer exchanged with IDTE (10 mM Tris, pH 8.0, 0.1 mM EDTA)+0.01% Tween-20 in Zeba™ Spin Desalting Columns, 40K MWC, 0.5 mL (Thermo Scientific, Inc), according to the manufacturer's protocol.

The single stranded amplicons were then made double-stranded as follows: 3× 50 μL aq. reactions were assembled containing 0.5 μM biotin-TEG-modified Primer A (5′-BiotinTEG/SEQ ID NO: 7), 22.5 μL of single stranded amplicon from the step above, and 25 μL Q5® High-Fidelity 2× Master Mix (New England Biolabs, Ipswich, Mass. USA). The reactions were activated at 98° C. for 60 s and then extended for 1 cycle (98° C. for 5 s, 65° C. for 60 s, 72° C. for 2 m). The resulting amplicon pool was purified from residual PCR components using the High Prep PCR system (MAGBIO GENOMICS, Gaithersburg, Md. USA) according to the manufacturer's protocol.

### Example 6: Expression of Polypeptide Comprising a Pro-Peptide vs. Mature-Peptide Coding Sequence

Template A (SEQ ID NO: 1, containing the coding sequence of SEQ ID NO: 2, and encodes for the polypeptide of SEQ ID NO: 3) was used in two separate PCRs to generate amplicons coding for either (a) the propeptide form of the wild-type protease of SEQ ID NO: 3 (amino acids 1-269), or (b) the mature form of wild-type protease of SEQ ID NO: 3 (amino acids 86-354). Forward Primer Al (SEQ ID NO: 10; 5′-CAGAA CGTCA CTCTC TCTTC ACTAA TACGA CTCAC TATAG GGAGA CCACA AGAAG GAGAT ATACA TATGG CTGAA GAAGC AAAAG AAAAA-3′) for the pro-form, and forward Primer A2 (SEQ ID NO: 11; 5′-CAGAA CGTCA CTCTC TCTTC ACTAA TACGA CTCAC TATAG GGAGA CCACA AGAAG GAGAT ATACA TATGG CGCAA TCGGT ACCAT GG-3′) were substituted for the propeptide form and the mature form, respectively, while Primer B (SEQ ID NO: 12; 5′-GCTCA CCTGG GCTAT GTATT AGTTA TTAAC GCGTT GCCGC TTCTG C-3′) was used in both reactions as the reverse primer. The reaction was activated at 98° C. for 30 s and then thermal cycled 24 times (98° C. for 5 s, 65° C. for 15 s, 72° C. for 30 s) followed by a final extension at 72° C. for 2 m. The resulting pro- amplicon or mature amplicon were purified from residual PCR components using the Agencourt AMPure XP system (Beckman Coulter, Inc., Indianapolis, Ind. USA) according to the manufacturer's protocol. A 34 μL aq. in vitro transcription/translation (IVTT) reaction was assembled on ice using the PURExpress® In Vitro Protein Synthesis Kit (New England Biolabs, Ipswich, Mass. USA). The IVTT reaction contained 13.6 pL PURExpress® Tube A, 2.72 μL PURExpress® Disulfide Bond Enhancer, 0.68 μL Murine RNase Inhibitor (#M0314), 9.86 μL PURExpress® Tube B, and 6.8 μL of the pro-amplicon or the mature amplicon. The IVTT reactions were incubated for 1 h at 16, 25, 30, and 37° C. Following incubation, the reactions were assayed for yield and activity of the expressed polypeptide.

Pro- and mature polypeptide yield was measured using a sandwich ELISA as follows: capture antibody specific to the polypeptide of SEQ ID NO: 3 was diluted 1:4000 in 11 mL of 1× PBS. 100 μL of this dilution was added to each well in a 96-well white Greiner Lumitrac 600 plate (E&K Scientific #25074) and incubated at 4° C. overnight. The following day, all plate wells were washed with 250 μL 1× TBS-T (Tris-Buffered Saline: 50 mM Tris, 150 mM NaCl +0.05% Tween-20) using a Tecan HydroFlex™ 3-in-1 microplate washer. 100 μL of Pierce Superblock Buffer (Thermo Fisher Scientific, Inc. #37353) was added to each well, and incubated at room temperature while shaking for 1 h at 800 rpm. Plates were washed as above to remove Superblock Buffer and unbound antibody. A standard curve of the polypeptide of SEQ ID NO: 3 was made at concentrations of 119, 59.5, 23.8, 9.5, 3.8, 1.5, 0.6, and 0.25 pg/μL in Ca-HEPES Buffer (50 mM HEPES+0.1 mM CaCl₂, pH 7.6) while the IVTT-expressed polypeptide was diluted 10× in 1× TBS-T+1 mM PMSF (Phenylmethanesulfonylfluoride dissolved in 100% Ethanol). 100 μL volumes of the standard curve in duplicate, and the IVTT samples in triplicate, were added to the plate to bind to the capture antibody. The plate was incubated at room temperature while shaking for 1 h at 800 rpm. Unbound reagent was removed by 3× wash with 250 μL 1× TBS-T as above. HRP-conjugated detection antibody specific to polypeptide of SEQ ID NO: 3 was diluted 1:8000 in 11 mL of 1× PBS, and 100 μL of this dilution was added to each well. The plate was incubated at room temperature while shaking for 1h at 800 rpm. Unbound detection antibody was removed by 3× wash with 250 μL 1× TBS-T as above. 11 mL of the working substrate for HRP, SuperSignal ELISA Pico Luminol Enhancer (Thermo Fisher Scientific, Inc. #37070), was made by diluting the Luminol Enhancer solution 1:1 with the provided Stable Peroxide Solution. 100 μL of this dilution was added to each well. Signal development was achieved by shaking for 1 m at 800 rpm. Total luminescence of each well was measured using a Biotek Synergy™ H1 microplate reader. A 5PL curve fit was applied to the standard curve in terms of Relative Luminescence Units (RLU). The yield of each sample was calculated using the curve fit from the standards in the same concentration range.

The activity of the IVTT-expressed polypeptide was measured using the EnzChek® Protease Assay Kit (Thermo Fisher Scientific, Inc. #E6638) as follows: the lyophilized substrate, called EnzChek-Green, was reconstituted to a concentration of 1 mg/mL with 200 μL 1× PBS and mixed by vortexing. In an amber microtube, this 1 mg/mL stock was diluted 1:25 with Ca-HEPES, for a 2× working stock at 40 ng/μL. A standard curve of the purified enzyme was made at concentrations of 595, 238, 95.2, 38.1, 15.2, 6.1, 2.4, and 1 pg/μL. IVTT samples were diluted 5× using Ca-HEPES. 10 μL of the EnzChek-Green protease substrate was aliquotted to each well in a 384-well black low volume plate (Corning #3676), and to this, 10 μL of the IVTT samples was immediately added in duplicate, and mixed by pipetting. The end dilution of the IVTT samples was 10×, and the in-assay concentration of EnzChek-Green was 20 ng/μL. Relative fluorescence units (RFU) were measured from each sample in intervals of 3 m over 35 m at 25° C. on the Biotek Synergy™ H1 with excitation/emission at 493/514 nm. The change in the RFU over time, “Mean Velocity,” of each point in the standard curve was calculated and a 5-PL curve fit was applied. The Mean Velocity of each sample was measured and the sample concentration was calculated using the curve fit from the standards in the same concentration range.

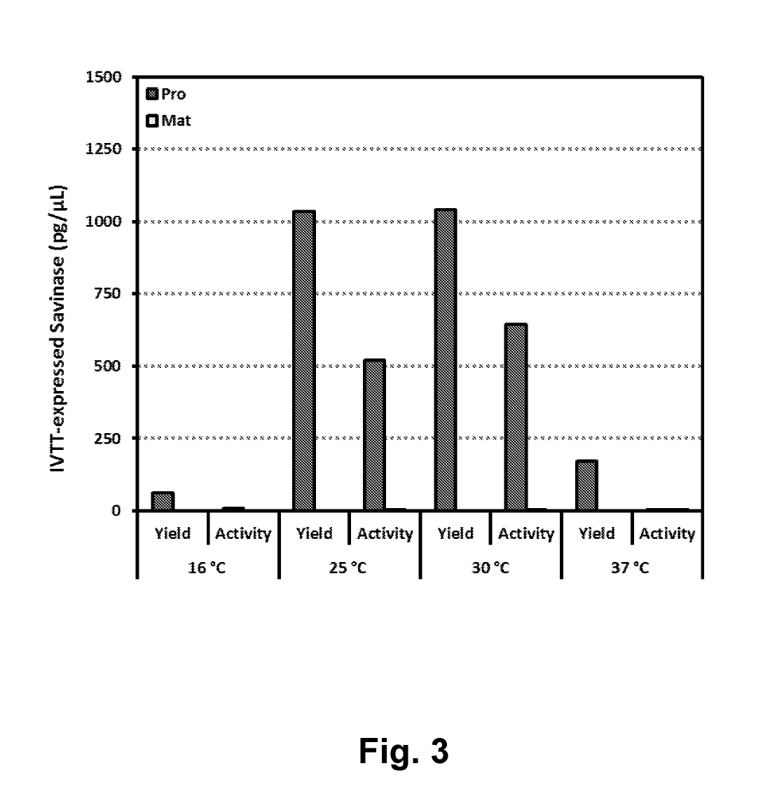

The yield and activity of IVTT-expressed pro- and mature protease are shown in FIG. 3. Notably, both yield and activity for the mature protease is considerably lower so low (barely visible on the chart) compared to the protease containing the propeptide.

### Example 7: Recovery of Released DNA Using Proximal vs. Distal Biotin Tags

Two separate PCRs were performed to generate DNA amplicons containing either a proximal or distal biotin tag. Both amplicons were generated from template A (SEQ ID NO: 4, containing the coding sequence of SEQ ID NO: 5, and encodes for the protease of SEQ ID NO: 6) as follows: a 50 μL aq. reaction was assembled containing 0.5 μM Primer A1 (SEQ ID NO: 13; 5′-CAGAA CGTCA CTCTC TCTTC AC-3′) and 0.5 μM TCO-biotin-TEG-modified Primer B1 (5′-TCO/Sp-C18/BiotinTEG/SEQ ID NO: 14; 5′-AAAAA ACGGA GCGAA CCACT TATC-3′) for the proximal biotin-tagged sequence; or 0.5 μM biotin-TEG-modified Primer A2 (5′-BiotinTEG/SEQ ID NO: 13) and 0.5 μM TCO-biotin-TEG-modified Primer B2 (5′-TCO/Sp-C18/BioTEG/SEQ ID NO: 14) for the distal biotin-tagged sequence, 1 pg of template A, and 25 μL Q5® High-Fidelity 2× Master Mix (New England Biolabs, Ipswich, Mass. USA). The reactions were activated at 98° C. for 30 s and then thermal cycled 27 times (98° C. for 5 s, 65° C. for 10 s, 72° C. for 20 s) followed by a final extension at 72° C. for 2 m. The biotinylated amplicons were purified from residual PCR components using the Agencourt AMPure XP system (Beckman Coulter, Inc., Indianapolis, Ind. USA) according to the manufacturer's protocol, and then conjugated to α-casein-coated microspheres as described supra.

For each proximal- and distal-biotinylated amplicon, two separate 250 μL aq. reactions were assembled on ice as follows: 232.8 μL of 2 mg/mL BSA dissolved in 50 mM HEPES, pH 7.6, 2 μg of random blocking DNA, 10.4 μL of purified Savinase enzyme at a concentration of 96 pg/μL, and 4.8 μL of either microsphere-bound amplicon. 125 μL from each reaction was immediately added to 375 μL of room temperature 3M Novec HFE-7500, 2% Pico-Surf 1 (The Dolomite Centre Ltd., Royston, UK) and emulsified as described in Example 4. The emulsions and the remaining aq. reaction volumes (referred to as the “input” for calculating % DNA recovery) were incubated for 1 h at 30° C. to digest the α-casein substrate and release the coupled amplicons. Following incubation, the aqueous fraction of each emulsion was extracted as described supra. The digested DNA in the input samples, and the recovered aqueous fractions were captured on streptavidin-coated beads as described supra, and diluted 100× with IDTE (10 mM Tris, pH 8.0+0.1 mM EDTA, Integrated DNA Technologies, Inc., Coralville, Iowa USA).

The concentration of captured DNA molecules was measured by qPCR as follows: a 15 μL aq. reaction was assembled containing 0.5 μM Primer A (SEQ ID NO: 15; 5′-GGCAT GCACG TTGCT AATTT-3′) and 0.5 μM Primer B (SEQ ID NO: 16; 5′-GCTAC AACAA GAACG CCTCT A-3′), 5 μL of diluted bead-bound DNA, and 7.5 μL SsoAdvanced™ Universal SYBR® Green Supermix (Bio-Rad, Hercules, Calif. USA). The reactions were activated at 95° C. for 30 s, thermal cycled 45 times (95° C. for 5 s, 60° C. for 5 s), followed by a melting curve measurement (95° C. for 5 s, 65° C. for 1 m, continuous increase to 95° C.), and finally cooled to 48° C. for 2 m. Thermal cycling and measurement of the SYBR® Green signal for the qPCR was performed using a LightCycler® 480 II (Roche, Basel, Switzerland).

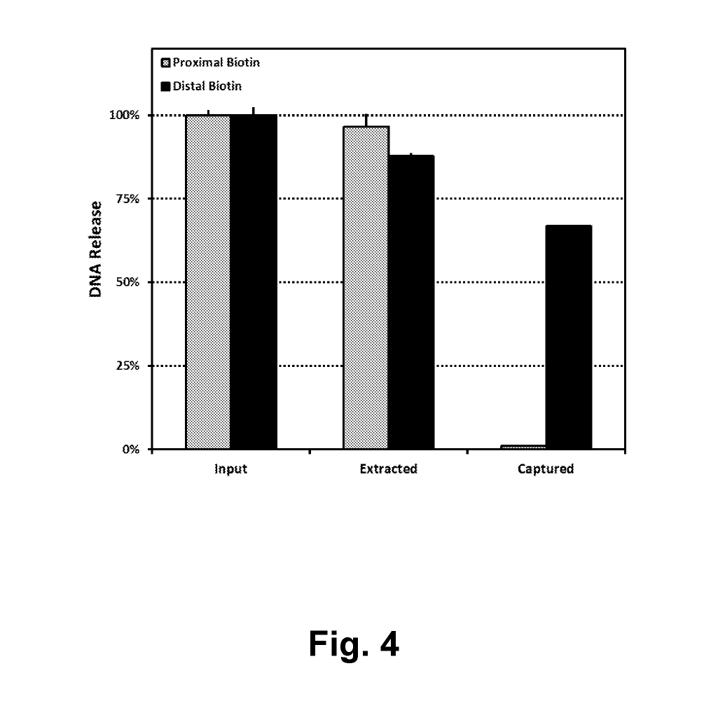

As shown in FIG. 4, the resulting differential capture from amplicons with a distal affinity tag was significantly higher compared to amplicons with a distal biotin affinity tag.

### Example 8: Emulsion Formation and Polypeptide Expression in the Presence of Ovoinhibitor

The procedures of Examples 1-4 were performed in the presence of ovoinhibitor. Ovoinhibitor was isolated from hen egg white following the method of Davis, Zahnley, and Donavan (1969, Biochemistry 8: 2044-2053) to a >75% purity, as estimated by SDS-PAGE. The preparation was confirmed to be nuclease-free use the DNaseAlert™ Kit (#11-02-01-04, Integrated DNA Technologies, Inc., Coralville, Iowa USA) according to the manufacturer's protocol.

Isolated ovoinhibitor was diluted in 50 mM HEPES and added to the IVTT reaction mixture and processed as described supra, with the following alterations: Microspheres conjugated to amplicon A and amplicon B were pooled such that approximately 20% were derived from SEQ ID NO: 1 (encoding for wild-type protease) and 80% were derived from SEQ ID NO: 4 (encoding for the catalytically inactive protease). In addition to the standard IVTT components in Example 4, the emulsion aqueous phase contains 0, 250, 500, 1000, 2000, 4000, or 8000 pg/μL ovoinhibitor. All emulsions were incubated for 2 h @ 30° C. then 6 h at 40° C. before the emulsions were broken in the presence of PMSF and recovered, as described in Example 5.

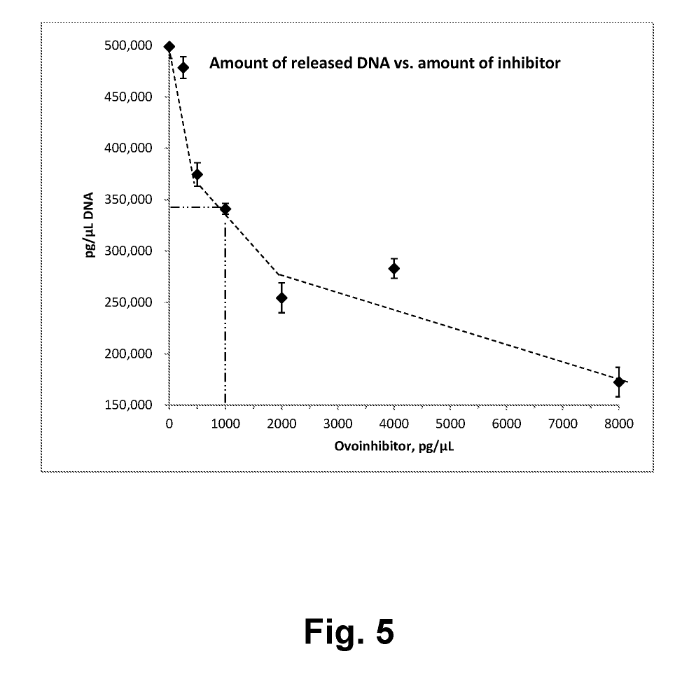

The amount of release amplicon A was measure by qPCR as follows: 15 μL aq. reactions were assembled containing 0.5 μM Primer A (SEQ ID NO: 15) and 0.5 μM Primer B (SEQ ID NO: 16), 5 μL of 100× diluted recovered DNA, and 7.5 μL SsoAdvanced™ Universal SYBR® Green Supermix (Bio-Rad, Hercules, Calif. USA). The reactions were activated at 95° C. for 30 s, thermal cycled 45 times (95° C. for 5 s, 60° C. for 5 s), followed by a melting curve measurement (95° C. for 5 s, 65° C. for 1 m, continuous increase to 95° C.), and finally cooled to 48° C. for 2 m. Thermal cycling and measurement of the SYBR® Green signal for the qPCR was performed using a LightCycler® II (Roche, Basel, Switzerland). As shown in FIG. 5, 1000 pg/μL ovoinhibitor reduces the amount of released amplicon A to 68% compared to the amount released in the absence of ovoinhibitor.

Although the foregoing has been described in some detail by way of illustration and example for the purposes of clarity of understanding, it is apparent to those skilled in the art that any equivalent aspect or modification may be practiced. Therefore, the description and examples should not be construed as limiting the scope of the invention.

1. A method of selecting for a polypeptide having protease activity, the method comprising:
suspending a plurality of synthetic compounds in an aqueous phase, wherein the synthetic compounds individually comprise:
(a) a polynucleotide encoding for a polypeptide;
(b) a protease substrate linked to said polynucleotide; and
(c) a selectable marker linked to said polynucleotide;
wherein the aqueous phase comprises components for expression of the polypeptide;
(ii) forming a water-in-oil emulsion with the aqueous phase, wherein the synthetic compounds are compartmentalized in aqueous droplets of the emulsion;
(iii) expressing the polypeptides within the aqueous droplets of the emulsion, wherein a polypeptide with protease activity in an aqueous droplet hydrolyzes the protease substrate in that droplet; and
(iv) separating the synthetic compounds to recover synthetic compounds comprising the protease substrate and/or synthetic compounds wherein the protease substrate has been hydrolyzed.

2. The method of claim 1, wherein the polypeptide comprises a propeptide.

3. The method of claim 1, wherein the plurality of synthetic compounds comprises at least about 10⁶ different synthetic compounds.

4. The method of claim 1, wherein the emulsion comprises at least about 10⁶ aqueous droplets/mL of emulsion.

5. The method of claim 1, wherein the aqueous droplets in the emulsion have an average diameter between about 0.05 μm and about 100 μm, inclusive.

6. The method claim 1 wherein the aqueous droplets in the emulsion have an average volume of between about 1 femtoliter and about 1 nanoliter, inclusive.

7. The method of claim 1, wherein the selectable marker is linked to the polynucleotide in a distal position relative to the protease substrate.

8. The method of claim 1, wherein the selectable marker is an affinity tag.

9. The method of claim 1, wherein the synthetic compounds individually comprise a solid phase.

10. The method of claim 9, wherein the solid phase is linked to said protease substrate, and wherein hydrolysis of the protease substrate releases the solid phase from the synthetic compound.

11. The method of claim 1, comprising separating the aqueous phase from the oil phase prior to step (iv).

12. A synthetic compound comprising:
(a) a polynucleotide encoding for a polypeptide;
(b) a protease substrate linked to said polynucleotide; and
(c) a selectable marker linked to said polynucleotide.

13. The synthetic compound of claim 12, wherein the polypeptide comprises a propeptide.

14. The synthetic compound of claim 12, wherein the selectable marker is linked to the polynucleotide in a distal position relative to the protease substrate.

15. The synthetic compound of claim 12, wherein the selectable marker is an affinity tag.

16. The synthetic compound of claim 12, further comprising a solid phase, wherein the solid phase is linked to said protease substrate.

17. A method of making the synthetic compound of claim 12, comprising:
(i) linking a protease substrate to a polynucleotide encoding for a polypeptide;
(ii) linking a selectable marker to the polynucleotide encoding for a polypeptide; and
(ii) recovering the synthetic compound.

18. A polynucleotide library comprising a plurality of different synthetic compounds according to claim 12.

19. A water-in-oil emulsion comprising the polynucleotide library of claim 18, wherein the synthetic compounds are compartmentalized in aqueous droplets of the emulsion.

20. A method of making the emulsion of claim 19, comprising:
(i) suspending the plurality of synthetic compounds in the aqueous phase; and
(ii) mixing the suspension of (i) with an oil.
